(19) United States
(12) Patent Application Publication
(10) Pub. No.: US 2026/0213013 A1
(43) Pub. Date: Jul. 23, 2026

(54) MULTIMODAL PATIENT HEALTH DATA ASSOCIATION AND RETRIEVAL WITH BINARY LATENT INDEXED MATRICES

(71) Applicant: Mayo Foundation for Medical Education and Research, Rochester, MN (US)

(72) Inventor: Hamid R. Tizhoosh, Rochester, MN (US)

(21) Appl. No.: 19/454,029

(22) Filed: Jan. 20, 2026

Related U.S. Application Data
(60) Provisional application No. 63/747,159, filed on Jan. 20, 2025.

(51) Int. Cl. G16H 50/20; G16H 10/20; G16H 10/60; G16H 15/00; G16H 30/40; G16H 70/00

(52) U.S. Cl. CPC G16H 50/20; G16H 10/20; G16H 10/60; G16H 15/00; G16H 30/40; G16H 70/00

(57) ABSTRACT

A binary matrix representation of multimodal patient health data is generated by inputting latent representations of the multimodal patient health data to a suitably trained machine learning model. Multimodal patient health data are accessed with a computer system. The multimodal patient health data include at least first patient health data of a first data type and second patient health data of a second data type. A first latent representation of the first patient health data and a second latent representation of the second patient health data are generated. The first and second latent representations are input to the machine learning model using the computer system, generating a binary matrix as an output. The binary matrix includes a concise representation of the multimodal patient health data. The binary matrix may then be outputted with the computer system.

### CROSS-REFERENCE TO RELATED APPLICATIONS

This application claims the benefit of U.S. Provisional Patent Application Ser. No. 63/747,159, filed on Jan. 20, 2025, and entitled “MULTIMODAL PATIENT HEALTH DATA ASSOCIATION AND RETRIEVAL WITH BINARY LATENT INDEXED MATRICES,” which is herein incorporated by reference in its entirety.

### BACKGROUND

Patient information is massive and complex, and is stored in different formats and in different archives (LIS, HIS, RIS, PACS, etc.). Exchanging patient information among units and clinics is difficult and patient matching for diagnostic and prognostic inference is practically impossible.

### SUMMARY OF THE DISCLOSURE

It is an aspect of the present disclosure to provide a method for generating a binary matrix representation of multimodal patient health data associated with a patient. The method includes accessing the multimodal patient health data with a computer system. The multimodal patient health data include at least first patient health data of a first data type and second patient health data of a second data type. A machine learning model is also accessed with the computer system, where the machine learning model is trained on training data to generate a binary matrix representation of patient health data from multimodal data. A first latent representation of the first patient health data is generated, and a second latent representation of the second patient health data is also generated. The first and second latent representations are input to the machine learning model using the computer system, generating a binary matrix as an output. The binary matrix includes a concise representation of the multimodal patient health data. The binary matrix may then be outputted with the computer system.

### BRIEF DESCRIPTION OF THE DRAWINGS

FIG. 1A is a diagram illustrating an example framework for multimodal data fusion. The framework integrates multimodal patient health data, such as the illustrated histopathology images and immunogenomic data, into a concise binary matrix representation, or patient code. The workflow encompasses unimodal transformation, multimodal latent association, and the generation of the binary matrix representation.

FIG. 1B is a diagram illustrating another example framework for multimodal data fusion. The framework in the illustrated example integrates multimodal patient health data, such as the illustrated histopathology images and textual patient health data, into a concise binary matrix representation, or lesion code. The workflow encompasses unimodal transformation, multimodal latent association, and the generation of the binary matrix representation.

FIG. 2 is a flowchart of an example method for generating a binary matrix representation of multimodal patient health data.

FIG. 3 is a flowchart of an example method for training a machine learning model (e.g., a neural network) to generate an embedding or other feature vector from a unimodal data subset of multimodal patient health data.

FIG. 4 is a flowchart of an example method for training a machine learning model (e.g., an autoencoder network) to generate a latent representation of a unimodal data subset of multimodal patient health data.

FIG. 5 is a flowchart of an example method for training a machine learning model (e.g., a neural network) to generate a binary matrix representation of multimodal patient health data from input latent representations of the multimodal patient health data.

FIGS. 6A-6D illustrate analysis of reconstructed features from multimodal association learning through hybrid autoencoders trained using histopathology images and immunogenomic data of lung and kidney datasets. The violin plots in FIGS. 6A and 6B illustrate the distribution of cosine similarity values between the original and reconstructed embeddings by the hybrid autoencoders, with the dashed line indicating the median. The accompanying bar plots provide a comprehensive overview of the mean squared error (MSE) across all original embeddings and their corresponding reconstructed counterparts. FIG. 6C illustrates the triplet loss during the training of MarbliX to learn a multimodal patient code of cases within each of lung and kidney datasets. FIG. 6D shows a heatmap illustrating the distinctions in the generated binary matrix representations among cases with various subtypes of lung and kidney cancers. The dissimilarity is expressed as the XOR of each monogram with every other monogram in the datasets.

FIGS. 7A-7H depict t-SNE maps depicting the distribution of modalities within a high-dimensional space. The visual representation showcases embeddings post-PCA transformation, specifically retaining the initial 64 components. The maps shows the relationship between cancer subtypes within lung (first row) and kidney (second row) datasets. The distributions of unimodal embeddings are presented in FIGS. 7A and 7E for the image modality, while FIGS. 7B and 7F illustrate the immunogenomics modality. Maps in FIGS. 7C and 7G show the distribution of real-value monogram representations, while FIGS. 7D and 7H display binary monograms, both generated by MarbliX.

FIG. 8 illustrates an example pipeline for utilizing MarbliX in a multimodal pathology search, aiming to identify similar cases to a given query case-exemplified here with a liver cancer patient.

FIGS. 9A-9H illustrate a comparative analysis of multimodal search performance. FIGS. 9A-9D depict MarbliX versus unimodal representations of histopathology images and immunogenomic data. Mean and standard deviation of macro average F1-score and accuracy for lung and kidney test folds. Leave-one-out and majority vote criteria are applied to retrieve top-1, MV@3, MV@5, and MV@10 for cases in the test folds. FIGS. 9E-9H depict multimodal search performance of real and binary MarbliX representations compared to unimodal representations of histopathology images and immunogenomic data. Diamond-shaped points represent the mean of macro-average precision and recall for lung and kidney test folds. Horizontal bars denote the standard deviation range. Leave-one-out and majority vote (MV) criteria are applied to retrieve top-1, MV@3, MV@5, and MV@10 for cases in the test folds.

FIG. 10 illustrates example binary matrix (e.g., patient code or monogram) representations generated using MarbliX for randomly selected patients with Lung Adenocarcinoma (LUAD) and Lung Squamous Cell Carcinoma (LUSC). The figure presents matrix bitwise XOR results: the left side illustrates {LUADset⊕LUADset}, the right side {LUSCset⊕LUSCset}, and the middle {LUADset⊕LUSCset}. Yellow pixels highlight features transitioning from 0 to 1, while purple pixels indicate the shift from 1 to 0. This color scheme serves to distinguish the presence or absence of features. In the context of {LUADset⊕LUSCset}, yellow pixels signify features unique to LUAD matrices, absent in LUSC, and purple pixels denote features specific to LUSC matrices, absent in LUAD.

FIG. 11 is a block diagram of an example multimodal association and retrieval with binary latent indexed matrix (MarbliX) system in accordance with some embodiments described in the present disclosure.

FIG. 12 is a block diagram of example components that can implement the system of FIG. 11.

FIG. 13 illustrates the visualization of example patient codes (or lesion codes) for well-differentiated, moderately-differentiated, and poorly differentiated squamous cell carcinoma cases.

### DETAILED DESCRIPTION

Described here are systems and methods for generating a machine readable code or pattern such as a barcode, a quick response (QR) code, or the like, that encodes patient information and disease information in a compact and non-traceable way. The machine readable code may be referred to as a “patient code.” The patient code can include a binary matrix or other machine readable code or pattern that encodes patient health data, such as radiology, pathology, and clinical disease information. Advantageously, the patient code can provide a safe and efficient summary of these patient health data to enable transmission and matching cases for uses in health consultations, telemedicine, and the like.

The disclosed systems and methods provide a multimodal framework for cancer and other clinical disease state diagnoses that integrates multimodal patient health data (e.g., histopathology images with immunogenomic sequencing data), and which generates from the multimodal data a binary patient code. In some implementations, the multimodal framework may be referred to as a Multimodal Association and Retrieval with Binary Latent Indexed matrix (MarbliX) framework.

Pre-existing multimodal artificial intelligence (AI) solutions lack the ability to incorporate multimodal patient health data of different data types (e.g., both histopathology and immunogenomic data). The disclosed systems and methods are capable of incorporating such multimodal patient health data types to unveil the underlying latent correlations between complex, yet informative modalities. The disclosed MarbliX framework harnesses the capabilities of deep learning to merge different data modalities (e.g., histopathology whole slide images (WSIs) with immune cell sequencing data), condensing them into a concise binary matrix representation, which may be referred to as a patient code or monogram. This compact binary matrix representation encapsulates the intricate patterns found within extensive multimodal data files. Moreover, the MarbliX framework serves as an efficient similarity search tool for establishing a comprehensive monogram-based archive. In this way, the MarbliX framework enables the matching and retrieval of similar cases to a given query case, thereby empowering clinicians and pathologists with an in-depth comprehension of cases. This facilitates more precise and timely diagnoses, reduces variability in assessments, and augments personalized treatment options.

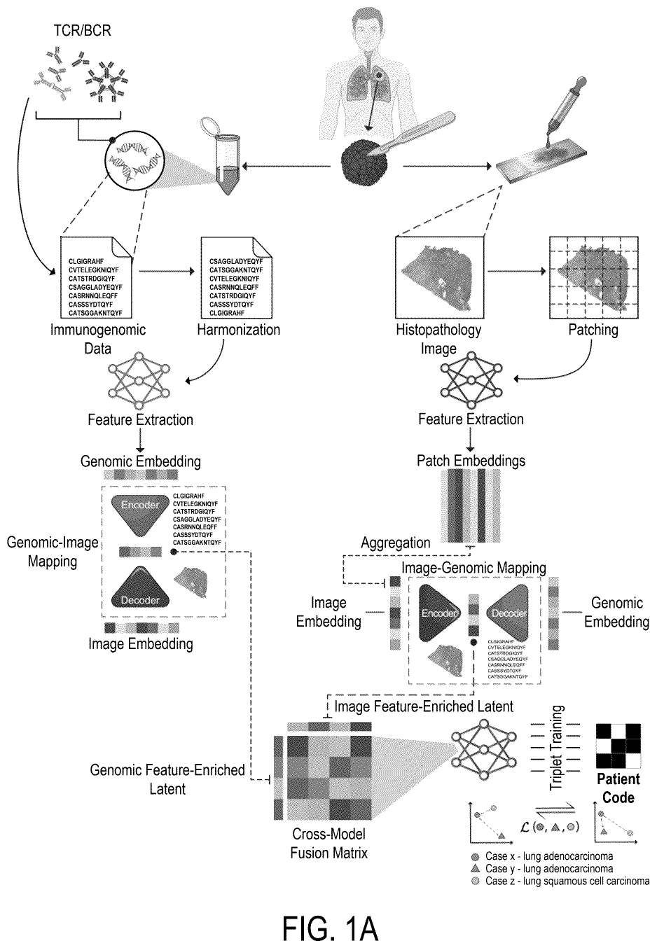

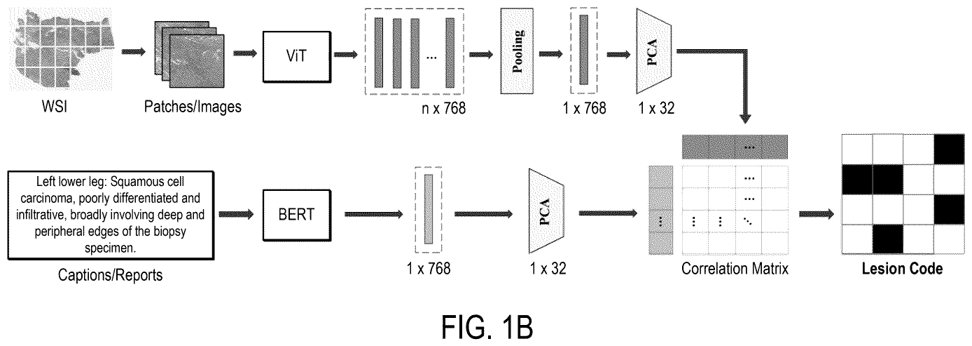

FIG. 1A illustrates an example workflow of a MarbliX framework for generating a binary patient code from multimodal patient health data. In this illustrated example, the multimodal patient health data include histopathology images and immunogenomic data. The design of the MarbliX framework involves three main phases: a unimodal transformation phase, a multimodal latent association phase, and monogram representation phase. The details of each of these phases is described below.

The integration of different data types in multimodal patient health data can be implemented by first aligning the different data types. By way of example, multimodal patient health data can include histopathology imaging data containing histopathology images and immunogenomic data containing immune cell sequencing data. Additionally or alternatively, the multimodal patient health data can include textual patient health data, such as pathology reports, clinical notes, captions describing the histopathology images, image annotations, radiology reports, laboratory result narratives, diagnostic interpretations, or other clinical documentation. These textual descriptions can be processed using a language model, such as a BERT model or other suitable language model, to generate feature vectors. These data types can be aligned to transform them into a common format, which enables joint manipulation and integration within a unified model. Hence, as a first stage, each modality or data type in the multimodal patient health data can be processed and transformed into a single feature vector or embedding. By way of example and for illustrative purposes, in the context of the notation (I, S), I can represent histopathology image data, while S can represent the immunogenomic data (e.g., a set of immune cell sequences) and/or textual patient health data of a given case.

To represent whole slide images (WSIs) from histopathology imaging data, the images can be divided into image patches. A collage can be formed for an image I after segmenting a tissue region from the background. As a non-limiting example, the image can be segmented using Otsu thresholding, or the like. This collage is a condensed representation, composed of a select set of representative patches extracted from the image, I, capturing the tissue characteristics that define the image. The collage can be generated by setting a similarity threshold to the 30th percentile, striking a balance between performance and computational/storage requirements. Once the collage is generated for the image, I, the deep features can be extracted from the individual patches that compose the collage. This process can be achieved by leveraging a pre-trained deep neural network, such as a DINO ViT network, which possesses the ability to extract patterns and meaningful information within these patches. As a result of this indexing, a feature vector, fᵢ, of image patch, Pᵢ, within the collage is generated by applying the neural network to the image patch.

In some implementations, hypocellular patches may be filtered out and discarded before feature extraction. Patches with low cellularity may not contain sufficient tissue characteristics to contribute meaningfully to the feature representation, and removing such patches can improve the quality of the resulting feature vectors.

To craft an all-encompassing feature vector that encapsulates the entirety of the image, I, and effectively represents its rich content, the average of the patch-level feature vectors can be computed, resulting in a single feature vector, f, that serves as a holistic representation of the entire image, I. Alternatively, median pooling can be used as a strategy to combine the patch-level feature vectors into a single feature vector, f. As a non-limiting example, a 768-dimensional embedding, or other feature vector, can be generated for an entire WSI.

For the immunogenomic data, raw RNA-seq files were utilized to reconstruct the immune repertoire of every subject. TRUST4 was employed to obtain the TCR and BCR sequences of each patient from their RNA-seq profiles. Rare sequences and artifacts were filtered out by excluding those that were not common to a sufficient proportion of subjects within each subtype class. Sequences that were not common to at least 30% of the patients within the subtype class were excluded, while a lower threshold (e.g., a threshold of 15%) can be applied to cases having a more limited number of samples available.

Before encoding the sequencing data into a dense feature vector, or embedding, a Seqwash method can be applied to preprocess the sequencing profiles and prepare them for feature extraction. Seqwash is a harmonization approach tailored to genetic sequencing data. The harmonization process prepares these sequences for analysis using deep neural network models designed for textual data by mitigating the effects of variability among subjects in terms of sequence lengths and unregulated sequence orders. Therefore, Seqwash was used to unify the patient profiles by aligning them into a standardized representation before proceeding with deep feature extraction. As a result, a single, coherent embedding or other feature vector that encapsulates vital information can be created while negating the effects of varying sequence orders within each subject's profile.

Applying Seqwash on sequencing profile S results in a harmonized set Sₕ. A pre-trained deep learning model G is then employed to distill features from the sequences by applying g=G(Sₕ), where g represents a feature vector, or embedding, extracted from the harmonized sequences set, Sₕ. As one non-limiting example, a large language model (LLM) or other language model can be used, such as a BERT model. As a non-limiting example, a 768-dimensional embedding can be generated.

Following the transformation of each modality into respective embeddings (e.g., histopathology image/into embedding f and immune cell sequence profile S into embedding g), the association between these embeddings is learned. However, as these embeddings were generated by different models they will have different ranges. In these instances, a min-max rescaling can be performed to bring the embeddings to a common scale before learning the association between them.

After normalization, association learning was performed by projecting the two embeddings into a shared latent space. In this shared space, the embeddings from both modalities coalesce to form a concise patient representation. This step addresses the issue of non-relevant features that may exist in the unimodal data obtained from a pretrained network. By merging these embeddings into an encoded representation, the pertinent features from each modality can be extracted and consolidated, thereby enhancing their combined synergy and informative value in subsequent analyses.

As a non-limiting example, the latent association can be implemented using the algorithm shown in Algorithm 1 below. In this example, two deep neural networks with an encoder-decoder (autoencoder) architecture are employed, each tailored to emphasize the salient features from its corresponding modality while suppressing extraneous information. The two hybrid models are trained to generate an encoded latent representation, highlighting the relevant features. Specifically, each autoencoder model is designed to take one modality and reconstruct the other, resulting in a latent representation that embodies the dominant features of its respective modality.

Algorithm 1: Multimodal Latent Association

| | |
|---|---|
| 1: | Input: |
| 2: | Histopathology image embedding f |
| 3: | Immune cell sequence profile embedding g |
| 4: | Training Stage: |
| 5: | Initialize [symbol] _{I} (Autoencoder for Histopathology): |
| 6: | while not converged do: |
| 7: | Forward pass: f → ε_{I}(f) → [symbol] _{I}(ε_{I}(f)) |
| 8: | Compute loss: l_{I} = MSE(g, [symbol] _{I}(ε_{I}(f))) |
| 9: | Backpropagate and update weights |
| 10: | Initialize [symbol] ₛ (Autoencoder for Immune Cell Sequences): |
| 11: | while not converged do: |
| 12: | Forward pass: g → εₛ(g) → [symbol] ₛ(εₛ(g)) |
| 13: | Compute loss: lₛ = MSE(f, [symbol] ₛ(εₛ(g))) |
| 14: | Backpropagate and update weights |
| 15: | Latent Representation: |
| 16: | Encode using the encoder of [symbol] _{I}: |
| 17: | u ← ε_{I}(f) |
| 18: | Encode using the encoder of [symbol] ₛ: |
| 19: | v ← εₛ(g) |
| 20: | return u, v |

In the first stage (illustrated in Algorithm 1, Lines 4-9), an autoencoder denoted as A_{I} is designed, where it takes the histopathology image embedding f as input, and reconstructs the immune cell sequence profile embedding, g. During training, the autoencoder A_{I} learns to focus on relevant features present in histopathology images that offer insights into immune cell sequence patterns. These features are encapsulated within the bottleneck layer, situated just before the first decoder layer of the autoencoder model, A_{I}.

Conversely, in the second stage (described in Algorithm 1, Lines 10-14), another autoencoder, denoted as A_{S}, is employed, which takes the immune cell sequence profile embedding, g, as input and reconstructs the histopathology image embedding, f. This design empowers the model to emphasize characteristics inherent to immune cell sequence data that are relevant to the histopathological context, also embedded within the bottleneck layer of the autoencoder model, A_{S}.

In a non-limiting example, both hybrid autoencoders A_{I} and A_{S} include an encoder with two dense layers of size 512 and 256, followed by a bottleneck layer of size 128, and finally, a decoder with two dense layers of size 256 and 512. All autoencoders were trained using the Adam optimizer and mean square error (MSE) as the loss function. In an example implementation, the image-genomics autoencoder was trained for 150 epochs with a learning rate of 1×10⁻⁵, while the genomics-image autoencoder was trained for 50 epochs with a learning rate of 1×10⁻⁴.

Following the training of the autoencoders, A_{I} and A_{S}, the encoder from each hybrid autoencoder is employed to generate an encoded latent for each sample, resulting in two encoded vectors: image features-enriched latent and genomic features-enriched latent (Algorithm 1, Lines 15-20). For instance, a latent representation, u, characterized by a strong emphasis on histopathological features, is derived through the application of A_{I} to the histopathological feature vector, f. Likewise, a latent representation, ν, accentuating immune cell characteristics, is derived by applying A_{S} to the sequencing feature vector, g. The resulting compact representation (size 128) not only reduces dimensionality but also encapsulates relevant aspects of both modalities. This representation serves as a powerful encoding of joint information extracted from histopathology images and immune cell sequences, facilitating profound integration in the subsequent phase.

After generating the two latent representations, u and ν, the next step within the MarbliX framework is the projection and indexing of these representations into a binary matrix, which may be a two-dimensional binary matrix referred to as a patient code or monogram. This binary matrix serves as an effective representation for capturing the intricate relationships and correlations that exist between the modalities of the multimodal patient health data. The process of learning the binary matrix representation enables a comprehensive exploration of the joint information encoded within the latent representations, u and ν. It will be appreciated that for multimodal patient health data containing more than two data types or modalities, more than two latent representations can be generated and used as a basis for generating a binary matrix (e.g., a higher dimensional binary matrix), or multiple binary matrices (e.g., multiple two-dimensional binary matrices representing different combinations of two latent representations of two data types).

To generate the binary matrix, a deep neural network is designed and trained to uncover the correlations between histopathology and immunogenomic features based on diagnosis. This process aims to unveil the underlying structure that interlinks histopathological characteristics with immune cell behavior among cases within the same diagnostic class. In other words, the deep neural network is engineered to capture the commonality in multimodal relationships among cases that share similar diagnoses, embedding these features within their respective binary matrices. Simultaneously, the deep neural network strives to discern the distinctions between cases of different diagnostic classes and to accentuate these disparities within their corresponding binary matrices. This is achieved by employing self-supervised training using a triplet loss to minimize the distance between patients with the same primary diagnosis and maximize the distance between patients with different primary diagnoses.

As described in Algorithm 2 below, matrix representation neural network includes three branches with shared weights, each taking a pair of latent representations u and ν. Thus, the neural network is provided with triplet pairs as input: {u, ν}, {u+, ν+}, and {u−, ν−}. The triplet pairs include an anchor pair a={u, ν}, a positive pair p={u+, ν+}, and a negative pair n={u−.ν−}. The anchor pair serves as the reference for which the neural network endeavors to generate a representative binary matrix. The positive pair shares the same diagnosis as the anchor pair, reinforcing common features. In contrast, the negative pair differs in diagnosis from the anchor pair, shedding light on the discrepancies between diagnostic classes.

The positive and negative pairs can be selected for each anchor pair by calculating pairwise Euclidean distances and identifying the farthest positive sample and the closest negative sample for each anchor. This approach can be implemented to ensure robust training by introducing hard triplets to the model, guiding toward learning the similarities between samples belonging to the same class, despite eventual dissimilarity between them. Analogously, this approach guides the model to generate different representations for cases that share common features in their data, but belong to different classes.

Algorithm 2: Learning Multimodal Monogram Representation

| | |
|---|---|
| 1: | Input: |
| 2: | u: image latent representation |
| 3: | v: sequencing latent representation |
| 4: | Training Stage: |
| 5: | Initialize [symbol] model with three branches [symbol] ₁, [symbol] ₂, [symbol] ₃ |
| 6: | [symbol] ₁, [symbol] ₂, [symbol] ₃ ← shared weights |
| 7: | for each triplet [symbol] ₁ (u, v), [symbol] ₂ (u⁺, v⁺), [symbol] ₃ (u⁻, v⁻) do: |
| 8: | M ← u ⊗ v |
| 9: | M̅ ← [symbol] (M) ← M |
| 10: | M̅_{binary} ← {if w > 0.5 then 1 else 0 for each w in M̅} |
| 11: | Calculate triplet loss: |
| 12: | d(a, p) ← distance(M̅, M̅⁺) |
| 13: | d(a, n) ← distance(M̅, M̅⁻) |
| 14: | [symbol] ₜᵣᵢₚₗₑₜ ← max{(d(a, p) − d(a, n)) + a, 0} |
| 15: | [symbol] ₁, [symbol] ₂, [symbol] ₃ ← update weights using gradient descent |

Within each branch (Algorithm 2, Lines 11-15), the pair of u and ν is projected into a matrix through the computation of the outer product between their respective layers. This tensor is then flattened and passed through three consecutive dense layers of size 1024, 256, and 64 to learn the deep multimodal relationship. The last layer of the model has a binary branch that generates a binary representation of the last layer. This enables the generation of compact binary representations, highly efficient for subsequent indexing and storage. As the tanh function results in values within the range of [−1, 1], the binary dense layer sets positive values to 1 and negative values to 0.

The following triplet loss function calculates the distances between the anchor's predicted matrix and both the positive and negative matrices:

$${L}_{triplet} ( a , p , n ) = max \{ d ( a , p ) - d ( a , n ) + α , 0 \} . \qquad ( 1 )$$

In Eqn. (1), a represents the anchor case, p represents a positive case (sharing the same diagnosis as the anchor), and n represents a negative case (having a different diagnosis from the anchor). The distance measure d(a, p) calculates the distance between the anchor and positive case matrices, while the distance measure d(a, n) calculates the distance between the anchor and negative case matrices. The margin parameter, α, ensures a minimum separation between the positive and negative cases. As a non-limiting example, the distance measure can use a Euclidean distance. Additionally or alternatively, the distance measures can use other distance metrics, such as an L1 norm, a Minkowski distance, a cosine similarity, or the like.

The matrix representation neural network, Q, can be trained for 150 epochs using the tanh activation function and Adam optimizer with a learning rate of 1×10⁻⁵. After training the matrix representation neural network, it can be utilized to generate binary matrices representations for new cases. This is done, for example, by applying a pair of latent representations to the neural network to generate the binary matrix. In a non-limiting example, the binary matrix may include an 8×8 binary matrix (with encoding capability to cover 264=1.8×10¹⁹ combinations) derived from the latent representations, u and ν, of the histopathology image and immune cell sequences, respectively.

In some implementations, an alternative unsupervised method can be used to generate the binary matrix representation without requiring training with triplet loss. In this approach, the feature vectors from two modalities (e.g., an image feature vector and a text feature vector) can be matrix multiplied to produce a correlation matrix. FIG. 1B illustrates another example workflow of a MarbliX framework for generating a binary patient code (also referred to as a lesion code) using this approach. In FIG. 1B, the white grid on the WSI thumbnail represents the selected tissue patches. The retained image patches are fed into a vision transformer (ViT) to generate deep feature vectors. These multiple feature vectors were then transformed into a single feature vector using a median pooling strategy. Concurrently, the corresponding text was input to a pretrained BERT model, resulting in a single feature vector. The text and image feature vectors were matrix multiplied, and the median of all the multiplied numbers was calculated. This median value served as a threshold. Any multiplied numbers below the median were set to zero, while numbers equal to or greater than the median were converted to one. This direct thresholding approach produces a binarized lesion code matrix. For example, when using 768-dimensional feature vectors, the resulting binary matrix can have dimensions of 768×768. This unsupervised binarization method provides a computationally efficient approach to generating binary matrix representations from multimodal data.

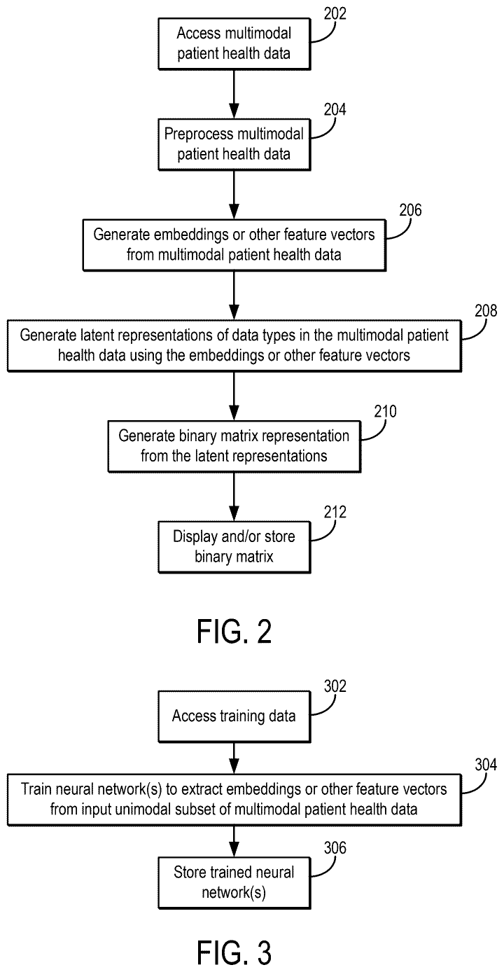

Referring now to FIG. 2, a flowchart is illustrated as setting forth the steps of an example method for generating a binary matrix representation of multimodal patient health data using suitably trained machine learning models. As will be described, the neural network or other machine learning algorithm takes latent representations of multimodal patient health data as inputs and generates a binary matrix representation of the multimodal patient health data as an output.

The method includes accessing patient health data with a computer system, as indicated at step 202. Accessing the patient health data may include retrieving such data from a memory or other suitable data storage device or medium.

In general, the patient health data are multimodal data that include more than one data type. As one example, the patient health data can include a data set corresponding to a first data type or modality and another data set corresponding to a second data type or modality. In a non-limiting example, the first data type can include histopathology data and the second data type can include immunogenomic data. In another non-limiting example, the first data type can include histopathology data and the second data type can include textual patient health data, such as pathology reports, clinical notes, captions describing the histopathology images, image annotations, radiology reports, laboratory result narratives, diagnostic interpretations, or other clinical documentation. More generally, the patient health data can include two or more data types selected from medical imaging data, histopathology data, genomic data, immunogenomic data, proteomic data, textual patient health data, clinical laboratory data, demographic data, or other such types of patient health data.

Medical imaging data may include images of the patient obtained with one or more different medical imaging modalities, including magnetic resonance imaging (MRI), computed tomography (CT), x-ray imaging, positron emission tomography (PET), ultrasound, and so on. The medical imaging data may also include parameters or features computed or derived from such images.

Histopathology data may include histopathology imaging data, clinical laboratory data, or the like. Histopathology imaging data can include digital pathology images, such as whole slide images (WSIs) of histopathology samples. The digital pathology images can include, for example, images of H&E slides, IHC slides, and the like. The histopathology data may also include data and/or information from pathology and radiology reports, which may be ordered by a physician during the course of diagnosis and treatment of various illnesses and diseases.

Examples of clinical laboratory data and/or histopathology data can include genetic testing and laboratory information, such as performance scores, lab tests, pathology results, prognostic indicators, date of genetic testing, testing method used, and so on.

In some instances, the patient health data can include one or more types of omics data, such as genomics data, immunogenomics data, proteomics data, transcriptomics data, epigenomics data, metabolomics data, microbiomics data, and other multiomics data types. The patient health data can additionally or alternatively include patient geographic data, demographic data, and the like. In some instances, the patient health data can include information pertaining to diagnoses, responses to treatment regimens, genetic profiles, clinical and phenotypic characteristics, and/or other medical, geographic, demographic, clinical, molecular, or genetic features of the patient.

Features derived from structured, curated, and/or EMR or EHR data may include clinical features such as diagnoses; symptoms; therapies; outcomes; patient demographics, such as patient name, date of birth, gender, and/or ethnicity; diagnosis dates for cancer, illness, disease, or other physical or mental conditions; personal medical history; family medical history; clinical diagnoses, such as date of initial diagnosis, date of metastatic diagnosis, cancer staging, tumor characterization, and tissue of origin; and the like. Additionally, the patient health data may also include features such as treatments and outcomes, such as line of therapy, therapy groups, clinical trials, medications prescribed or taken, surgeries, radiotherapy, imaging, adverse effects, and associated outcomes.

Patient health data can include a set of clinical features associated with information derived from clinical records of a patient, which can include records from family members of the patient. These clinical features and data may be abstracted from unstructured clinical documents, EMR, EHR, or other sources of patient history. Such data may include patient symptoms, diagnosis, treatments, medications, therapies, responses to treatments, laboratory testing results, medical history, geographic locations of each, demographics, or other features of the patient which may be found in the patient's EMR and/or EHR. The patient health data may also include measurement data collected from wearable devices (e.g., physiological measurements or other data recorded with a wearable device). Physiological measurements that may be recorded with a wearable device include heart rate, temperature, or other physical parameters. Other data that may be recorded with a wearable device may include measurements or stress, measurements of sleep state and/or sleep quality, and the like. Additionally, the patient health data may include location data and/or environmental factors.

As a non-limiting example, epigenomics data may include data associated with information derived from DNA modifications that are not changes to the DNA sequence and regulate the gene expression. These modifications can be a result of environmental factors based on what the patient may breathe, eat, or drink. These features may include DNA methylation, histone modification, or other factors which deactivate a gene or cause alterations to gene function without altering the sequence of nucleotides in the gene.

Microbiomics data may include, for example, data derived from the viruses and bacteria of a patient. These features may include viral infections which may affect treatment and diagnosis of certain illnesses as well as the bacteria present in the patient's gastrointestinal tract which may affect the efficacy of medicines ingested by the patient.

Proteomics data may include data associated with information derived from the proteins produced in the patient. These features may include protein composition, structure, and activity; when and where proteins are expressed; rates of protein production, degradation, and steady-state abundance; how proteins are modified, for example, post-translational modifications such as phosphorylation; the movement of proteins between subcellular compartments; the involvement of proteins in metabolic pathways; how proteins interact with one another; or modifications to the protein after translation from the RNA such as phosphorylation, ubiquitination, methylation, acetylation, glycosylation, oxidation, or nitrosylation.

As a non-limiting example, genomics data can be extracted from blood or saliva samples collected from individuals. A deep phenotypic characterization of these individuals can be assembled. For instance, an analysis using whole exome sequencing (“WES”) can be completed.

Demographic data may include patient age, patient gender, patient race, and/or patient ethnicity.

As one non-limiting example, patient health data can include clinical features associated with information derived from clinical records of a patient, which in some instances can also include records from family members of the patient. These clinical features and data may be abstracted from unstructured clinical documents, EMR, EHR, or other sources of patient history. Such data may include patient symptoms, diagnosis, treatments, medications, therapies, responses to treatments, laboratory testing results, medical history, geographic locations, environmental factors, demographics, or other features of the patient which may be found in the patient's EMR and/or EHR.

Additionally or alternatively, patient health data can include clinical features derived from structured, curated, EMR and/or EHR data, such as diagnoses; symptoms; therapies; outcomes; patient demographics, such as patient name, date of birth, gender, and/or ethnicity; diagnosis dates for illness, disease, or other physical or mental conditions; personal medical history; family medical history; clinical diagnoses, such as date of initial diagnosis; and the like. Additionally, the patient health data may also include features such as treatments and outcomes, such as line of therapy, therapy groups, clinical trials, medications prescribed or taken, surgeries, imaging, adverse effects, and associated outcomes.

In some implementations, the multimodal patient health data, or subsets of the data contained within the multimodal patient health data, can be preprocessed, as indicated at step 204. By way of example, when the multimodal patient health data includes imaging data (e.g., medical imaging data, histopathology imaging data), the images can be preprocessed to generate image patches from the images contained in the imaging data. These image patches may be combined into a collage or other collection that provides a condensed representation of the image patches, as described above.

Additionally or alternatively, when the multimodal patient health data includes sequencing data (e.g., genomic data, immunogenomic data, proteomic data), the sequencing data (e.g., RNA-seq files) can be processed to filter out rare sequences, artifacts, and the like. For instance, rare sequences and artifacts can be filtered out by excluding those that were not common to a sufficient proportion of subjects within each subtype class. Additionally or alternatively, the sequencing data can be preprocessed to harmonize the sequences in the sequencing data to overcome the impact of variability among subjects in terms of sequence lengths and unregulated sequence orders. For instance, the sequences in the sequencing data can be aligned into a standardized representation.

Feature vectors, or embeddings, are then generated for each data type or modality represented in the multimodal patient health data, as indicated at step 206. For example, when the multimodal patient health data include two data types (e.g., histopathology imaging data, immunogenic data), a first feature vector can be generated from the first data type (e.g., histopathology imaging data) in the multimodal patient health data and a second feature vector can be generated from the second data type (e.g., immunogenic data) in the multimodal patient health data. As noted above, the first and second feature vectors may be first and second embeddings.

In general, the feature vectors may be extracted or otherwise generated from the multimodal patient health data by applying the respective data subset from the multimodal patient health data to a suitable machine learning model. For example, a first data type from the multimodal patient health data can be applied to a first machine learning model to generate the first feature vector, or first embedding, and a second data type from the multimodal patient health data can be applied to a second machine learning model to generate the second feature vector, or second embedding. As one non-limiting example, the first machine learning model may be a first neural network and the second machine learning model may be a second neural network.

As a non-limiting example, when the first data type includes imaging data (e.g., medical imaging data, histopathology imaging data), the first neural network may include a neural network that is trained to extract patterns and/or other meaningful information from images or image patches. The first neural network may include a vision transformer network, such as DINO-ViT, to generate feature vectors from the imaging data.

In some implementations, the imaging data may include a plurality of image patches. The image patches can be individually applied to the first neural network to generate a feature vector, or embedding, for each image patch. In some implementations, hypocellular patches may be filtered out and discarded before feature extraction. Patches with low cellularity may not contain sufficient tissue characteristics to contribute meaningfully to the feature representation, and removing such patches can improve the quality of the resulting feature vectors. The resulting patch-level feature vectors can then be combined to generate the first feature vector. As an example, the average of the patch-level feature vectors can be computed to generate the first feature vector.

As a non-limiting example, when the second data type includes sequencing data (e.g., genomic data, immunogenomic data, proteomic data), the second neural network may include a neural network that is trained to extract patterns and/or other meaningful information from sequencing data. The second neural network may include a language model (e.g., a large language model (LLM)), such as BERT, to generate feature vectors from the sequencing data.

Thus, in the foregoing implementations, the first neural network and the second neural network may each be accessed with the computer system and the first data type and second data type of the multimodal patient data applied to the respective ones of the first and second neural networks. Accessing a trained neural network may include accessing network parameters (e.g., weights, biases, or both) that have been optimized or otherwise estimated by training the neural network on training data. In some instances, accessing the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed.

An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs.

A latent representation is then generated for each data type or modality represented in the multimodal patient health data, as indicated at step 208. These latent representations can be generated from the feature vectors for the respective data types in the multimodal patient health data. For example, when the multimodal patient health data include two data types (e.g., histopathology imaging data, immunogenic data), a first latent representation can be generated from the first feature vector associated with the first data type (e.g., histopathology imaging data) in the multimodal patient health data and a second latent representation can be generated from the second feature vector associated with the second data type (e.g., immunogenic data) in the multimodal patient health data.

As a non-limiting example, the first latent representation can be generated using a first autoencoder network that has been trained on training data to reconstruct a feature vector associated with the second data type from the first feature vector. When the first data type is imaging data, the first latent representation can include an image features-enriched latent vector. The first latent representation can be generated by applying the first feature vector to the first autoencoder network and extracting the first latent representation from a layer of the first autoencoder network. For example, the first latent representation can be extracted from a bottleneck layer of the first autoencoder network.

Additionally or alternatively, the second latent representation can be generated using a second autoencoder network that has been trained on training data to reconstruct a feature vector associated with the first data type from the second feature vector. When the second data type is sequencing data, the second latent representation can include a sequencing features-enriched latent vector. The second latent representation can be generated by applying the second feature vector to the second autoencoder network and extracting the second latent representation from a layer of the second autoencoder network. For example, the second latent representation can be extracted from a bottleneck layer of the second autoencoder network.

Thus, in the foregoing implementations, the first autoencoder network and the second autoencoder network may each be accessed with the computer system and the first feature vector and second feature vector applied to the respective ones of the first and second autoencoder networks. Accessing a trained autoencoder network may include accessing network parameters (e.g., weights, biases, or both) that have been optimized or otherwise estimated by training the autoencoder network on training data. In some instances, accessing the autoencoder network can also include retrieving, constructing, or otherwise accessing the particular autoencoder network architecture to be implemented. For instance, data pertaining to the layers in the autoencoder network architecture (e.g., number of layers, type of layers, ordering of layers, connections between layers, hyperparameters for layers) may be retrieved, selected, constructed, or otherwise accessed.

A binary matrix representation of the multimodal patient health data is then generated by applying the first latent representation and the second latent representation to a neural network that has been trained to generate binary matrix representations from input latent representations, as indicated at step 210. As described above, the neural network can be trained using a self-supervised learning approach with a triplet loss function. By applying the first and second latent representations to the trained neural network, a binary matrix is generated. The binary matrix, which may also be referred to as a patient code or monogram, can include a binary matrix or other machine readable code or pattern that encodes multimodal patient health data and clinical disease information.

Thus, in the foregoing implementations, the matrix representation network may be accessed with the computer system and the first and second latent representations applied to the matrix representation network. Accessing a trained matrix representation network may include accessing network parameters (e.g., weights, biases, or both) that have been optimized or otherwise estimated by training the neural network on training data. In some instances, accessing the matrix representation network can also include retrieving, constructing, or otherwise accessing the particular network architecture to be implemented. For instance, data pertaining to the layers in the matrix representation network architecture (e.g., number of layers, type of layers, ordering of layers, connections between layers, hyperparameters for layers) may be retrieved, selected, constructed, or otherwise accessed.

The binary matrix generated by inputting the latent representations of the multimodal patient health data to the matrix representation network can then be displayed to a user, stored for later use or further processing, or both, as indicated at step 212.

Advantageously, the binary matrix can provide a safe and efficient summary of these multimodal patient health data and clinical disease information to enable transmission and matching cases for uses in health consultations, telemedicine, and the like. Thus, as one example the binary matrix can be used in search and retrieval tasks. For example, the binary matrix can be used to query a database to retrieve the analogous binary matrices generated for other patients and which share similar patterns with the query. This feature enhances the discoverability of relevant cases and expedites the decision making process in healthcare.

Referring now to FIG. 3, a flowchart is illustrated as setting forth the steps of an example method for training one or more neural networks on training data, such that the one or more neural networks are trained to receive a unimodal data subset of multimodal patient health data as an input in order to generate an embedding, or other feature vector, of that unimodal data as an output.

In general, the neural network can implement any number of different neural network architectures. For instance, the neural network could implement a convolutional neural network, a residual neural network, or the like. As described above, when the unimodal data include imaging data, the neural network may be implemented as part of a vision transformer model, such as a DINO ViT model. Additionally, when the unimodal data include sequencing data, the neural network may be implemented as part of an LLM or other language model, such as a BERT model. Alternatively, the neural network could be replaced with other suitable machine learning or artificial intelligence algorithms, such as those based on supervised learning, unsupervised learning, deep learning, ensemble learning, dimensionality reduction, and so on.

The method includes accessing training data with a computer system, as indicated at step 302. Accessing the training data may include retrieving such data from a memory or other suitable data storage device or medium. In general, the training data can include unimodal data corresponding to the data type for which the neural network is trained to generate embeddings or other feature vectors. Additionally, the training data may include other data, such as embeddings or other feature vectors associated with the unimodal data in the training data. In some embodiments, the training data may include unimodal data and/or embeddings that have been labeled (e.g., labeled as containing patterns, features, or characteristics indicative of a clinical disease state; and the like).

The method can include assembling training data from the unimodal data and/or embeddings using a computer system. This step may include assembling the unimodal data and/or embeddings into an appropriate data structure on which the neural network or other machine learning algorithm can be trained. Assembling the training data may include assembling unimodal data, embeddings, and/or other relevant data.

One or more neural networks are then trained on the training data, as indicated at step 304. In general, the neural network can be trained by optimizing network parameters (e.g., weights, biases, or both) based on minimizing a loss function. As one non-limiting example, the loss function may be a contrastive loss function. For instance, the training process can include applying network pairs of similar and dissimilar data points and adjusting the network weights to minimize the distance between similar pairs while maximizing the distance between dissimilar pairs in the embedding space.

The one or more trained neural networks are then stored for later use, as indicated at step 306. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

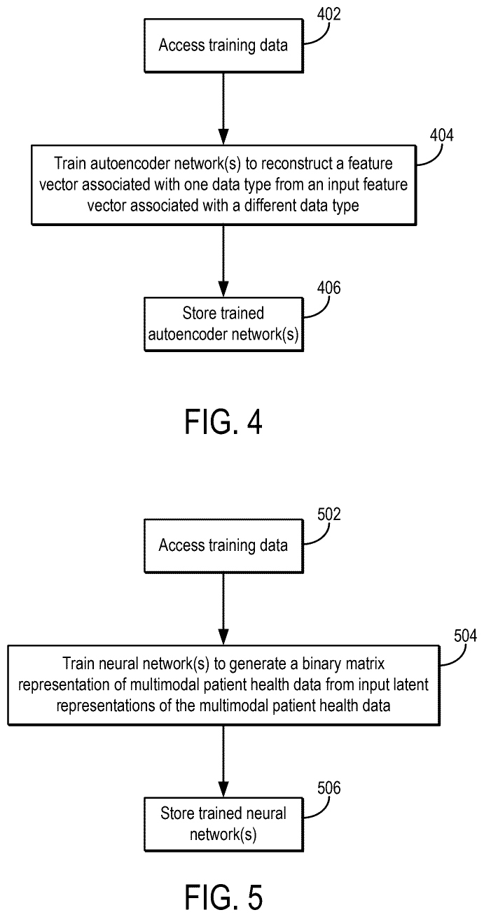

Referring now to FIG. 4, a flowchart is illustrated as setting forth the steps of an example method for training one or more autoencoder networks on training data, such that the one or more autoencoder networks are trained to receive a feature vector of a first data type as an input in order to reconstruct a feature vector of a second data type as an output.

The method includes accessing training data with a computer system, as indicated at step 402. Accessing the training data may include retrieving such data from a memory or other suitable data storage device or medium. In general, the training data can include feature vectors associated with different data types or modalities. For example, the training data can include first feature vectors associated with a first data type and second feature vectors associated with a second data type. The first data type can be associated with the input to the autoencoder network and the second data type can be associated with the reconstructed output of the autoencoder network. In some embodiments, the training data may include feature vectors that have been labeled (e.g., labeled as containing patterns, features, or characteristics indicative of a clinical disease state; and the like).

The method can include assembling training data from the first and second feature vectors using a computer system. This step may include assembling the first and second feature vectors into an appropriate data structure on which the autoencoder network can be trained. Assembling the training data may include assembling feature vectors and other relevant data.

One or more autoencoder networks are then trained on the training data, as indicated at step 404. In general, the autoencoder network can be trained by optimizing network parameters (e.g., weights, biases, or both) based on minimizing a loss function. As one non-limiting example, the loss function may be an MSE loss function.

In a non-limiting example, the autoencoder network can include an encoder with two dense layers of size 512 and 256, followed by a bottleneck layer of size 128, and then a decoder with two dense layers of size 256 and 512. The autoencoder network can be trained using the Adam optimizer and MSE as the loss function.

The one or more trained autoencoder networks are then stored for later use, as indicated at step 406. Storing the autoencoder network(s) may include storing network parameters (e.g., weights, biases, or both), which have been computed or otherwise estimated by training the autoencoder network(s) on the training data. Storing the trained autoencoder network(s) may also include storing the particular network architecture to be implemented. For instance, data pertaining to the layers in the autoencoder network architecture (e.g., number of layers, type of layers, ordering of layers, connections between layers, hyperparameters for layers) may be stored.

Referring now to FIG. 5, a flowchart is illustrated as setting forth the steps of an example method for training one or more neural networks on training data, such that the one or more neural networks are trained to receive latent representations as inputs in order to generate a binary matrix representation as an output.

The method includes accessing training data with a computer system, as indicated at step 502. Accessing the training data may include retrieving such data from a memory or other suitable data storage device or medium. In general, the training data can include first and second latent representations associated with respective first and second data types or modalities. Additionally, the training data may include other data, such as clinical disease state information associated with the patient whose multimodal patient health data were used to generate the latent representations. Thus, in some embodiments, the training data may include first and second latent representations that have been labeled (e.g., labeled as containing patterns, features, or characteristics indicative of a clinical disease state; and the like).

The method can include assembling training data from the latent representations using a computer system. This step may include assembling the latent representations into an appropriate data structure on which the neural network can be trained. Assembling the training data may include assembling latent representations and other relevant data. For instance, assembling the training data may include generating labeled data and including the labeled data in the training data. Labeled data may include latent representations that have been labeled as belonging to, or otherwise being associated with, one or more different classifications or categories. For instance, labeled data may include latent representations that have been labeled as being associated with one or more clinical disease states.

One or more neural networks are then trained on the training data, as indicated at step 504. In general, the neural network can be trained by optimizing network parameters (e.g., weights, biases, or both) based on minimizing a loss function. As described above, the loss function may be a triplet loss function. In these instances, the training data can be assembled into triplet pairs, such as anchor pairs, positive pairs, and negative pairs, as described above in more detail. In an example implementation, the matrix representation neural network can be trained for 150 epochs using a tanh activation function and Adam optimizer with a learning rate of 1×10⁻⁵.

The one or more trained neural networks are then stored for later use, as indicated at step 506. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

The disclosed MarbliX framework enables fusing the subtle morphological characteristics found in histopathology images with the intricate immune response patterns within immune cell sequencing profiles. The underlying rationale for this choice is deeply rooted in the clinical implications of these modalities. Histopathology data, with its potentials to unveil the subtleties of tissue structures, and immune cell sequencing data, capable of unraveling the molecular nuances of immune responses.

In an example study, the MarbliX framework was implemented and evaluated using datasets obtained from The Cancer Genome Atlas (TCGA), which included histopathology images and genomic data. Specifically, cases from two primary sites, lung and kidney, were sourced from TCGA. Only cases featuring both WSI and genomic profiles were considered. This resulted in a dataset comprising 535 lung adenocarcinoma (LUAD) cases and 510 lung squamous cell carcinoma (LUSC) cases for the lung primary site. For the kidney primary site, the dataset consisted of three classes: 508 kidney renal clear cell carcinoma (KIRC) cases, 248 kidney renal papillary cell carcinoma (KIRP) cases, and 38 kidney chromophobe (KICH) cases. MarbliX was evaluated using 5-fold cross validation for lung dataset while 2-fold cross validation was used to evaluate kidney dataset due to the limited number of cases in the KICH subtype as enough cases from every subtype is needed for meaningful training.

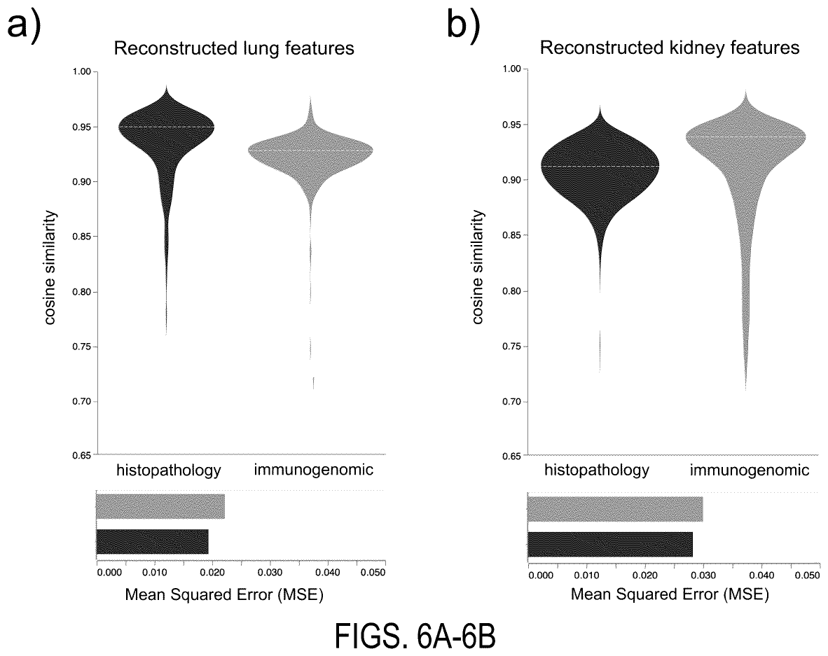

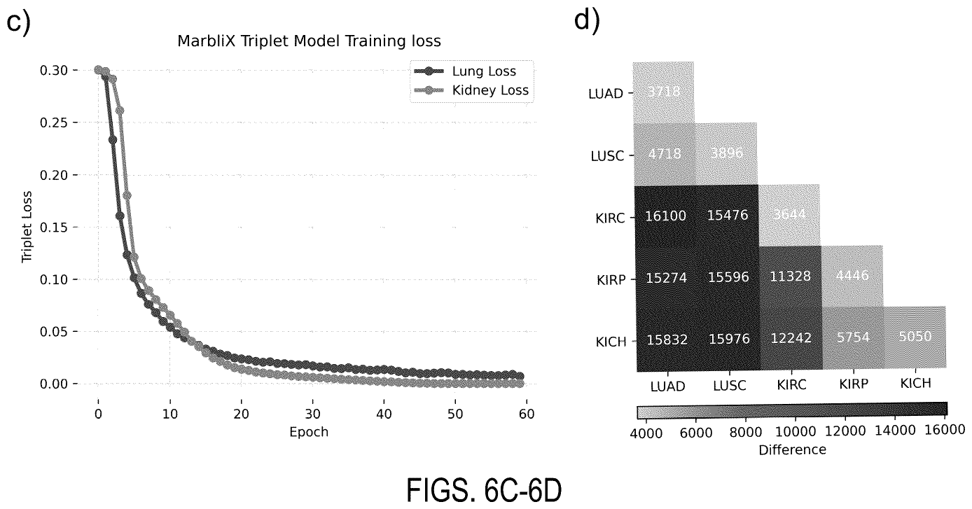

Different experiments were conducted to evaluate the quality of the patient representation. An experiment was performed to analyze the quality of the multimodal latent association applied through the hybrid autoencoders to map histopathology features to immunogenomic features, and vice versa. As shown in FIGS. 6A-6D, the cosine similarity was calculated between every histopathology and immunogenomic embedding extracted by the pretrained model with the reconstructed histopathology and immunogenomic, respectively, embeddings by the trained hybrid autoencoders. The violin plots in FIGS. 6A and 6B show the distribution of the cosine similarities between the original embedding and the reconstructed embedding of every case in the test sets of lung dataset (FIG. 6A) and kidney dataset (FIG. 6B). The median cosine similarities of 0.95 for lung histopathology and 0.91 for kidney histopathology indicate a strong overall resemblance between the original and reconstructed embeddings, suggesting effective feature retention. Similarly, in the immunogenomic distribution, with median cosine similarities of 0.93 for lung and 0.94 for kidney, the model maintains a high degree of similarity on average during the reconstruction process. The tightly packed quartiles in both lung and kidney histopathology plots imply consistent performance across the majority of cases.

In FIGS. 6A and 6B, the accompanying bar plots provide a quantitative assessment of the reconstruction quality by illustrating the average MSE between the original embeddings and their reconstructed counterparts for both lung and kidney datasets. The, the MSE values exhibit consistency across both histopathology and immunogenomics reconstructions, ranging between 0.020 and 0.030 for both datasets. The MSE values indicate that the model performs well in minimizing the squared differences between the original and reconstructed embeddings, reaffirming the reliability of the reconstruction process for both histopathological and immunogenomic features in the lung and kidney datasets.

The training curves shown in FIG. 6C provide an overview of the learning curves of the final MarbliX models trained on triplet loss, aiming to learn monogram representations for lung and kidney cases. The curves represent the evolution of the loss function over training epochs for each model. Initially, both models exhibit a sharp decrease in the loss, indicative of effective learning and convergence tendency. The model trained on the kidney dataset demonstrates a slightly more gradual decline in the loss compared to its lung counterpart. This might suggest that the kidney dataset poses greater complexity or variability, requiring the model to adapt more cautiously.

The binary monograms generated by MarbliX were analyzed to assess both inter and intra-dissimilarity between the representations. A set of 19 random cases was selected from the test set of each subtype. Quantifying the dissimilarity between matrices involved performing a bitwise XOR operation to identify differing bits between any two matrices. The resulting heatmap, displaying calculated dissimilarities, is presented in FIG. 6D. The diagonal of the heatmap reflects the smallest dissimilarity, indicating that MarbliX generates similar representations for patients with shared diagnoses.

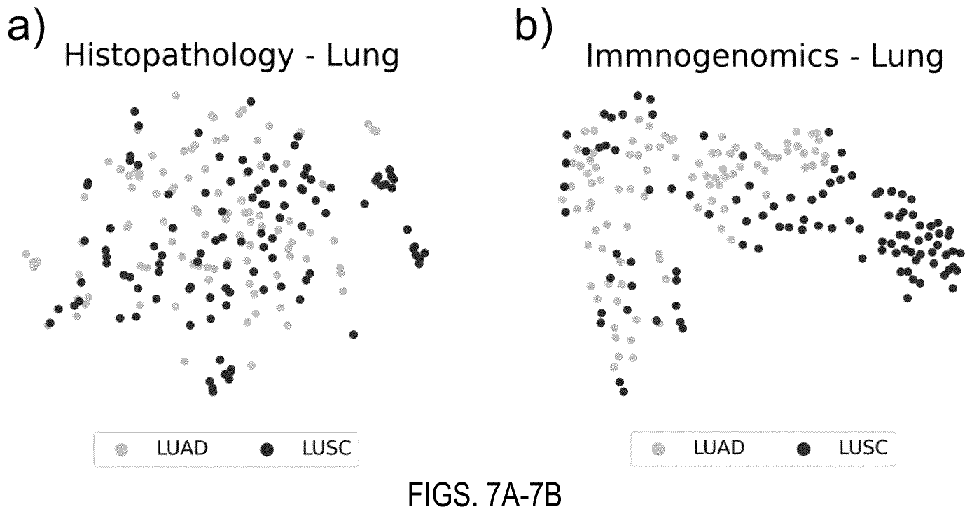

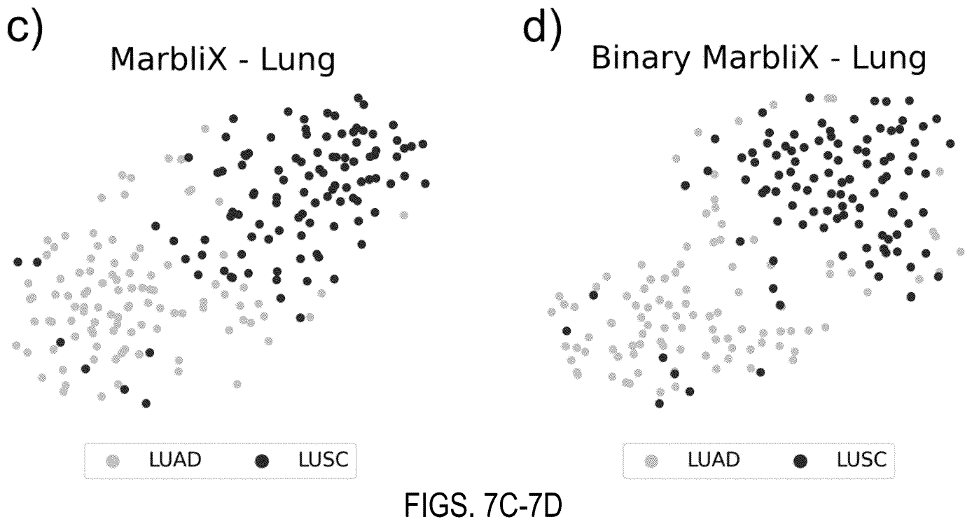

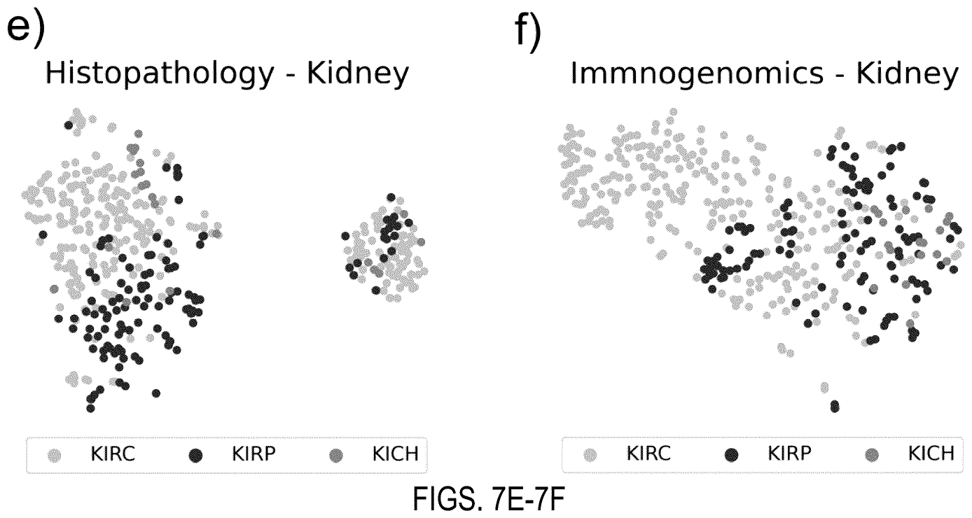

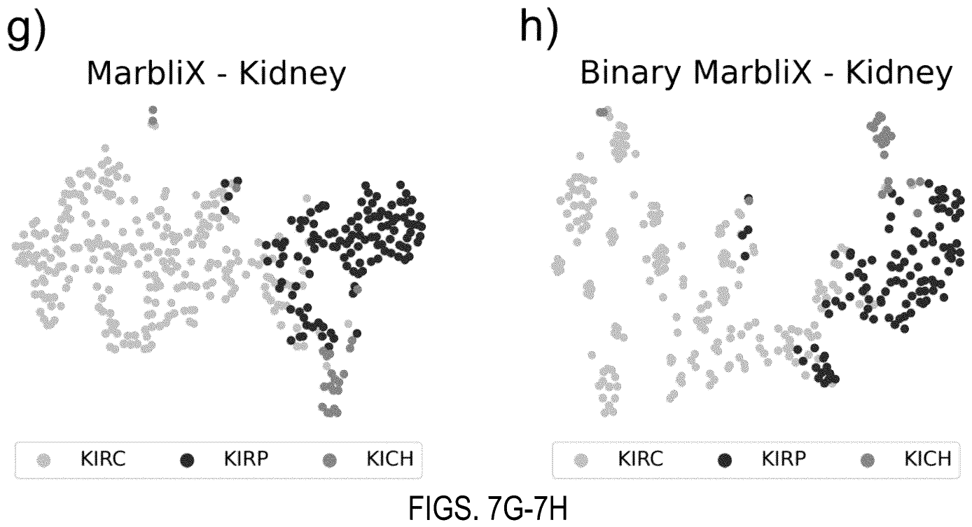

In assessing the efficacy of MarbliX in generating high quality multimodal patient representations, an experiment was conducted, comparing them to unimodal representations derived from histopathology and immunogenomics data. Principal component analysis (PCA) transformation was applied to extract the top 64 components from each embedding in the lung and kidney test sets, using folds not employed during training. These components were then projected into a high-dimensional space, forming the t-SNE maps presented in FIGS. 7A-7H. The maps provide comprehensive insights into feature properties, highlighting distinctions among various subtype classes. In FIG. 7A, lung histopathology image embeddings of LUAD and LUSC are intermixed, lacking a clear discriminative pattern. FIG. 7B demonstrates a more discriminative pattern in immunogenomic embeddings, although some LUSC features intertwine with LUAD features. Merging histopathology and immunogenomics features into a monogram using MarbliX enhances distribution in FIGS. 7C and 7D, showcasing real and binary features. Notably, the t-SNE maps reveal improved separation of LUAD and LUSC, particularly using binary monogram representations.

Transitioning to kidney maps, FIG. 7E shows two well-separated clusters of mixed cased from the three kidney subtypes in the histopathology t-SNE map. Analysis reveals that this separation is influenced by different hospitals, indicating the dominance of WSI technical properties in the embeddings. Immunogenomic t-SNE map (FIG. 7F) exhibits a more meaningful distribution than histopathology, with KIRC being the most discriminated class due to the available number of cases during training compared to KIRP and KICH. Despite this, MarbliX application refines the distribution of patient representations (FIGS. 7G and 7H), creating distinct groups for the three kidney cancer subtypes, highlighting the ability of MarbliX to derive compact yet discriminative representations from diverse modalities.

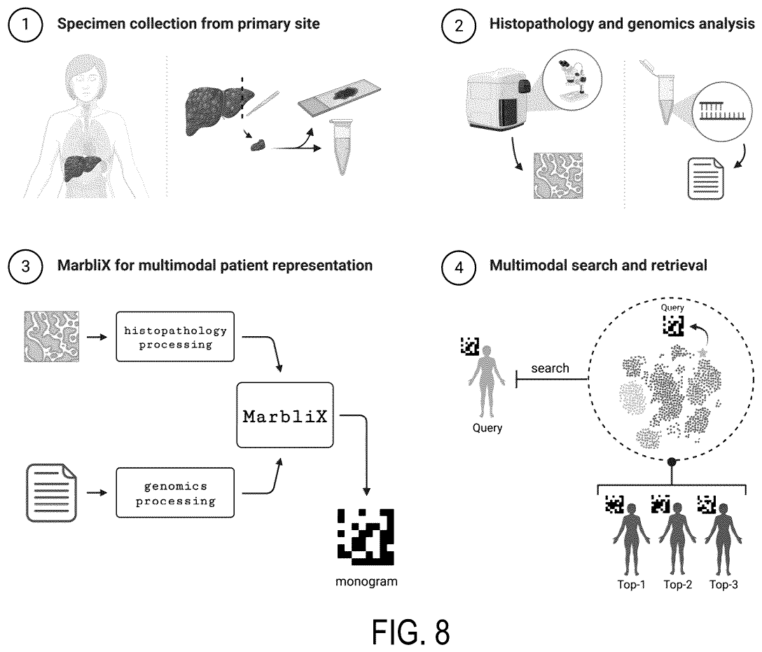

MarbliX demonstrated utility through the application of the binary matrix representation for conducting efficient multimodal search and retrieval. As illustrated in FIG. 8, the monogram, a condensed binary matrix representation, integrates diverse features extracted from multimodal data, thereby streamlining the process of multimodal search and retrieval. MarbliX was employed to fuse each patient's histopathology image and immunogenomic data into a monogram. The process begins with specimen collection for histopathology staining and genomics analysis. In the second stage, the tissue slide is digitized, and RNA sequencing is conducted to obtain the patient's whole slide image and genomics profile, respectively. In the third stage, following unimodal transformations of the histopathology image and genomics profile, MarbliX integrates the two modalities into a binary multimodal representation, referred to as “monogram”. The final stage involves a multimodal search using the generated monogram as a query to identify the top n (in this case, n=3) similar matrices in the archive. The search can be performed through Hamming distance comparison. The retrieved matrices can be utilized to identify the similar patients' medical information, including histopathology images, genomics data, treatment history, and risk stratification.

Advantageously, the binary matrix representation enables bidirectional search capabilities, allowing searches to be performed in multiple directions. For example, the binary matrix representation can be used to search for similar images using text as a query, to search for similar text or reports using images as a query, or to search for similar patients using a combination of image and text data as a query. This bidirectional search functionality enhances the versatility of the multimodal search and retrieval system.

To assess MarbliX's performance, “leave-one-out” validation was performed, utilizing monograms generated by MarbliX for cases in the test folds of lung and kidney datasets. Cases in the test folds were not part of the training set. The evaluation involved searching for similar cases within the monograms archive, treating each case as a query case by excluding it from the search process. The majority vote criterion was applied for top-3 (MV@3), top-5 (MV@5), and top-10 (MV@10) retrievals, requiring at least n/2+1 of the top-n retrieved cases to belong to the same diagnosis class as the query case.

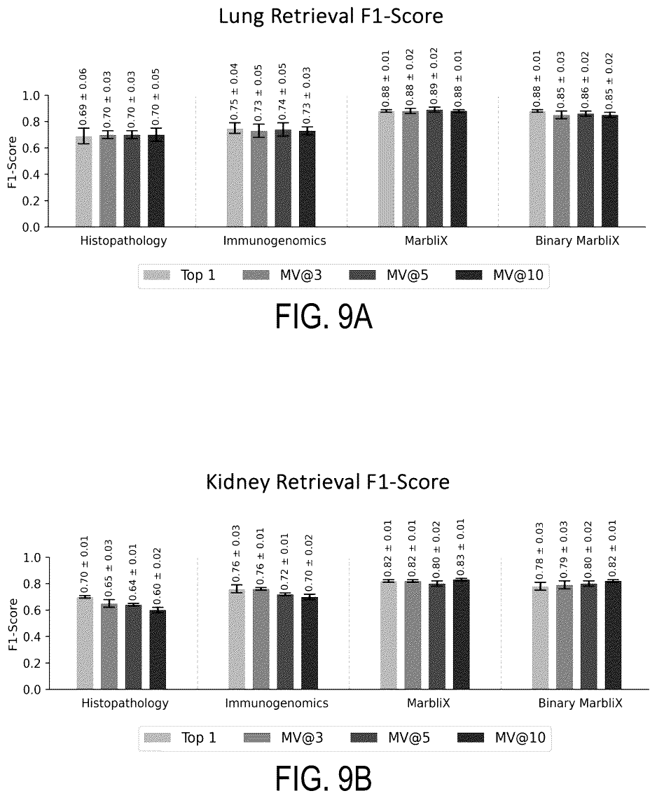

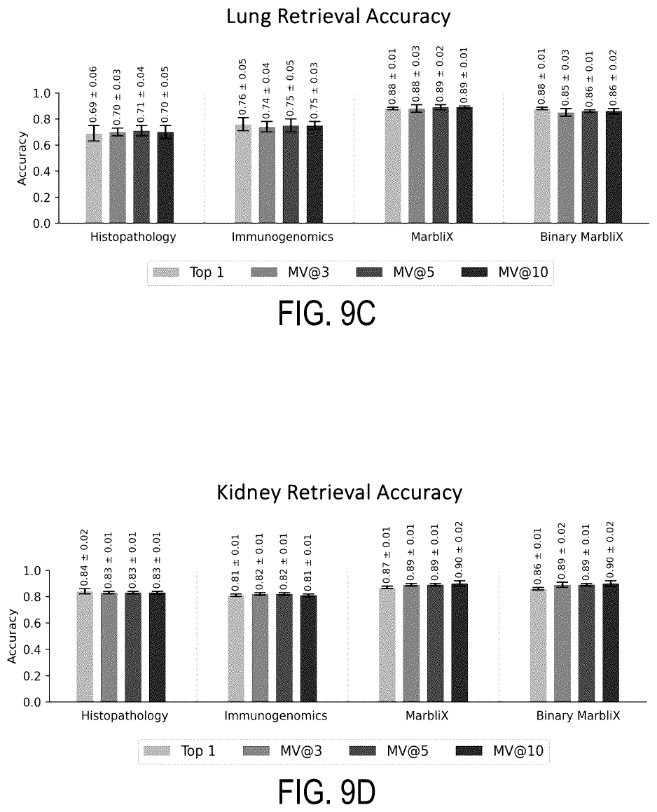

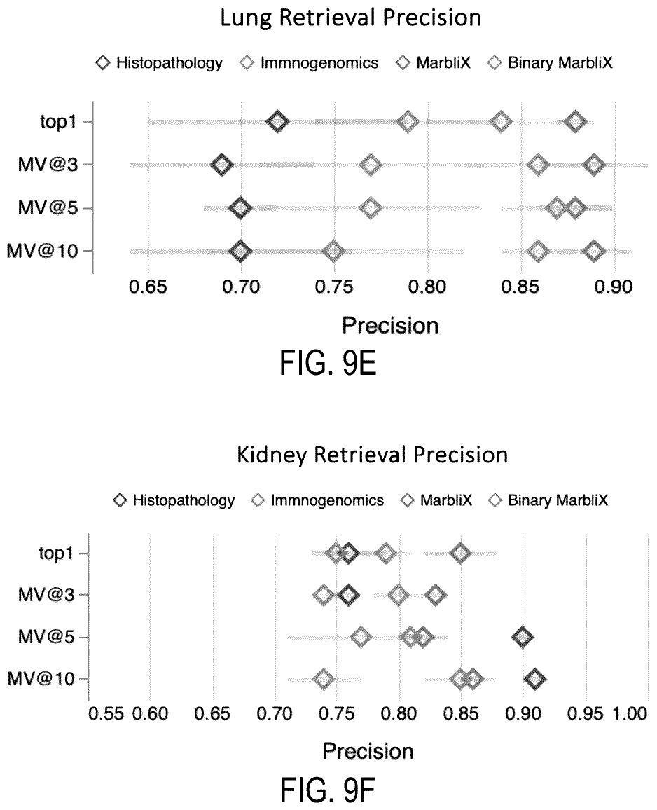

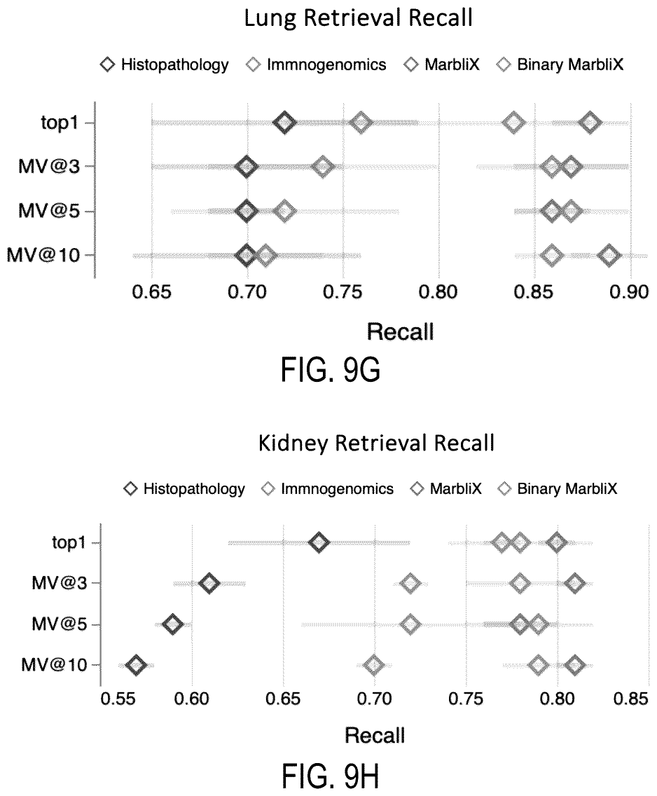

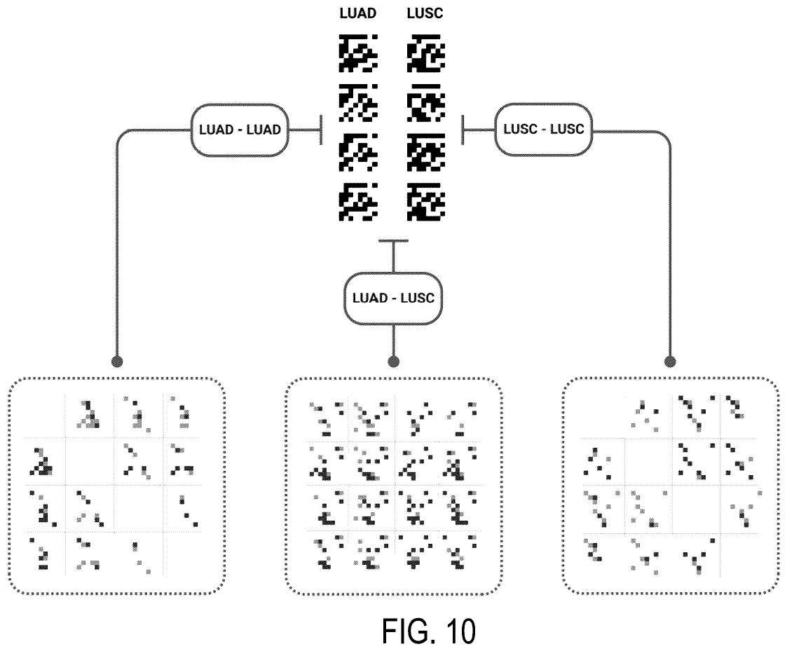

FIGS. 9A-9H present a comparative analysis of multimodal search performance. FIGS. 9A-9D presents the mean and standard deviation of macro average F1-score and accuracy for the test folds. The results of MarbliX representations, real and binary monograms are compared against unimodal representations of histopathology and immunogenomics. Analyzing the lung dataset results in FIGS. 9A and 9C, multimodal search using monogram yields more accurate and similar retrieved cases compared to unimodal searches. MarbliX consistently achieves performance between 85% and 89% across all criteria from top-1 to MV@10, surpassing the 69%-71% and 73%-76% ranges of histopathology and immunogenomics, respectively. The integration of distinctive patterns from both modalities into a compact multimodal representation notably enhances the quality of retrieved cases. Analyzing the performance on the kidney dataset shown in FIGS. 9B and 9D, MarbliX representation also out-performs unimodal representations. The best performance was achieved using real monograms, with F1-score ranging between 80% and 83%, and accuracy between 87% and 90%. However, employing binary monograms results in a slight decrease in the F1-score range (78%-82%). Immunogenomic representations outperform histopathology representations in terms of F-score (70%-76% vs. 60%-70%), while accuracy remains comparable. Notably, all kidney representations exhibit lower macro average F1-score values compared to accuracy values, reflecting dataset imbalance.

In FIGS. 9E-9H, the precision and recall results are illustrated for the same search evaluation on the lung and kidney datasets. The mean precision and mean recall of the folds are represented with the diamond shape, while the standard deviation of each mean value is shown as a horizontal bar. As shown in the figure, MarbliX representation maintains more stable results across the folds (different test sets) compared to the unimodal representations. This is evident from the generally shorter standard deviation bars for both lung and kidney results, indicating a more consistent performance. Furthermore, MarbliX maintains higher precision and recall compared to both lung histopathology and immunogenomic representations. For the kidney dataset, although the histopathology representation achieved the highest precision when a top-5 and top-10 majority vote was applied (90%-91%), it also achieved the lowest recall for the same measures (56%-59%). Binary MarbliX representation resulted in approximately the same recall as immunogenomics over the top-1 measure but demonstrated higher precision. Overall, MarbliX consistently maintained precision and recall above 78% for top-1, MV@3, MV@5, and MV@10 measures.

In summary, MarbliX demonstrates robust performance in multimodal search and matching, leveraging monograms to fuse histopathology and immunogenomic data effectively. Overall, MarbliX maintained more accurate and stable performance compared to unimodal representations.

As described above, the MarbliX framework can be used to generate multimodal binary matrix representations that are similar for patients with similar characteristics and distinct for those with different attributes. In an example study, similarity was defined based on a shared diagnosis, specifically the commonality of cancer subtypes. FIG. 10 illustrates MarbliX's efficacy in achieving this objective. The figure presents four LUAD monograms and four LUSC monograms generated for randomly selected samples from each class. It is evident that matrices from patients with the same subtype exhibit a consistent pattern (intra-similarity).

To highlight this intra-similarity and inter-dissimilarity, FIG. 10 displays the result of applying bitwise XOR on each LUAD matrix with every other matrix within the LUAD set. The same process was applied to the LUSC set, resulting in {LUSCset−LUSCset}. Additionally, the set of LUAD was XORed with the set of LUSC to identify the differences between each LUAD matrix and every LUSC matrix in the sets. Yellow pixels represent values turning from 0 to 1, while purple indicates the shift from 1 to 0. This color scheme is particularly informative when analyzing the results of {LUADset−LUSCset}, where yellow pixels signify features present in LUAD matrices but absent in LUSC matrices, and purple denotes features present in LUSC matrices but absent in LUAD matrices.

As shown in FIG. 10, the dissimilarity within LUAD or LUSC matrices is notably lower than the dissimilarity resulting from XORing the two sets. This is evident from the greater number of white empty areas in the intra-XOR results, as opposed to the more densely colored squares in the {LUADset−LUSCset} difference.

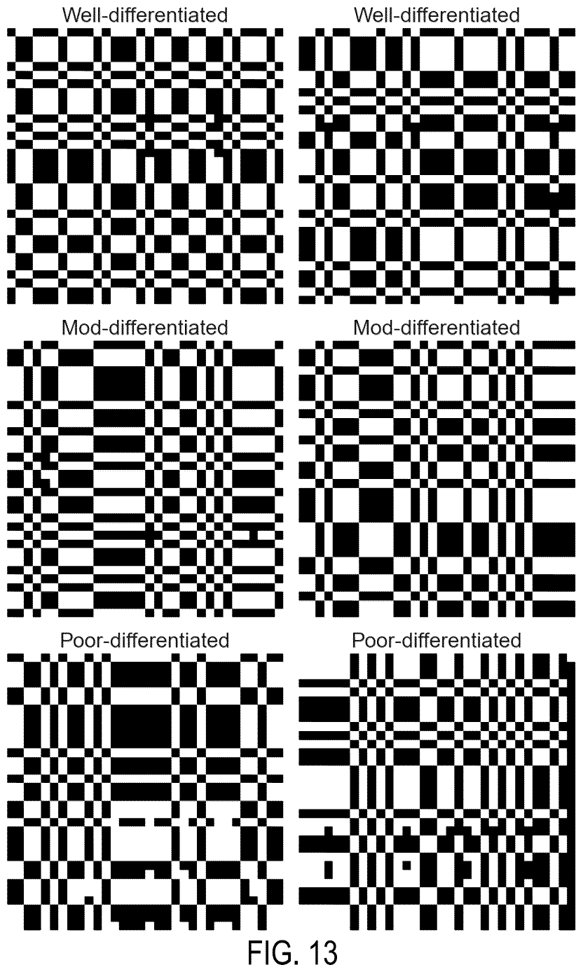

In some implementations, the binary matrix patterns generated by the MarbliX framework may correlate with clinical characteristics of the patient's disease state. For example, the binary matrix representations may visually distinguish between different tumor differentiation grades, such as well-differentiated, moderately-differentiated, and poorly-differentiated tumors, as illustrated in FIG. 13. Cases with similar differentiation grades may exhibit similar patterns in their binary matrix representations, while cases with different differentiation grades may exhibit distinct patterns. This correlation between binary matrix patterns and clinical characteristics can facilitate the identification and classification of disease states based on the generated binary matrix representations.

In some implementations, the binary matrix patterns generated by the MarbliX framework can correlate with clinical characteristics of the patient's disease state. For example, the binary matrix representations may visually distinguish between different tumor differentiation grades, such as well-differentiated, moderately-differentiated, and poorly-differentiated tumors, as illustrated in FIG. 13. Cases with similar differentiation grades may exhibit similar patterns in their binary matrix representations, while cases with different differentiation grades may exhibit distinct patterns. By way of non-limiting example, well-differentiated cases may exhibit relatively uniform and regular alternating patterns with consistent small-scale structures throughout the matrix, while moderately-differentiated cases may display increased irregularity with larger contiguous regions, and poorly-differentiated cases may demonstrate the most irregular patterns with substantial regions of solid areas and less consistent organization.

The correlations between binary matrix patterns and clinical characteristics can be leveraged to identify and classify disease states using various techniques. For instance, machine learning classifiers, such as convolutional neural networks, support vector machines, or random forest classifiers, can be trained on labeled binary matrix representations to automatically classify new cases based on their differentiation grade or other clinical characteristics. Additionally or alternatively, pattern matching techniques can be employed to compare a query binary matrix against a database of reference matrices with known clinical characteristics, using similarity metrics such as Hamming distance to identify the most similar cases and infer the clinical characteristics of the query case. Statistical analysis of the binary matrix patterns, such as measuring the distribution of contiguous regions, the frequency of specific sub-patterns, or the overall entropy of the matrix, can also provide quantitative features that correlate with disease states and can be used for classification purposes. This correlation between binary matrix patterns and clinical characteristics can facilitate the identification and classification of disease states based on the generated binary matrix representations.

The motivation for this research work stems from the significant challenges and unexplored opportunities that arise due to the conspicuous scarcity of multimodal solutions adept at merging histopathology images with genetic sequencing data in general, and with immunogenomic data in particular. Furthermore, as far as knowledge extends, the fusion of histopathology images with immunogenomic data remains an untapped area. By fusing these data modalities within the MarbliX framework, one objective is to effectively bridge critical research gaps and contribute to a more comprehensive understanding of cancer cases, while also unlocking new avenues for potential research in this domain. The work at hand brings forth four substantial contributions to the burgeoning field of multimodal research.

First and foremost, MarbliX introduces an approach that condenses diverse data modalities into a concise representation, referred to as binary matrix representation, patient code, or monogram. This concept enables the generation of a binary matrix representation for each individual patient, serving as a tangible representation of their multimodal data. This representation encapsulates the intricate patterns unearthed from various data sources, thereby affording each patient a highly informative imprint or signature. This approach empowers researchers and medical practitioners to extract rich insights from a patient's multimodal clinical data, facilitating personalized and data-driven healthcare.

An additional contribution of the MarbliX framework lies in its ability to create a binary matrix representation of multimodal patient data, which often comprises voluminous files like histopathology whole slide images (WSIs) and genetic sequencing data. This compactness holds can advantageously improve time efficiency and reduce computational complexity and storage requirements. Employing monograms in diverse downstream tasks such as search and retrieval not only streamlines these operations but also unlocks a powerful tool that can handle data in varying formats, sizes, complexities, and sources.

Furthermore, another advantage of the MarbliX framework is its backbone independence, a feature that distinguishes it as a versatile multimodal AI framework. The capability to map different modalities within a latent space not only contributes to the model's training efficiency but also enables the extraction of associated patterns from data modalities originating from diverse sources within the medical field. This adaptability is paramount for addressing the heterogeneous nature of medical data.

As yet another advantage, the MarbliX framework serves as a valuable asset in facilitating multimodal search, an important task in medical research and diagnosis. With the ability to construct monogram-indexed archives, MarbliX empowers practitioners with an interactive tool to multimodal search for cases similar to a given query. This process involves generating a monogram for the query case and then scouring the archive to retrieve the most analogous monograms that share similar patterns with the query. This feature enhances the discoverability of relevant cases and expedites the decision-making process in healthcare.

The evaluation of MarbliX has demonstrated its success in capturing multimodal patient representations by integrating histopathology images and immunogenomic data to generate monogram representations. These monograms serve as condensed binary representations of patients' multimodal features, achieved through unimodal processing and transformation, multimodal latent association by training hybrid autoencoders, and self-supervised training with triplet loss. The assessment of patient representation demonstrated the efficacy of MarbliX in capturing distinctive features from both histopathology and immunogenomics modalities.

The similarity analysis of generated monograms showed intra-similarity for patients with the same primary diagnosis and inter-dissimilarity between those with different primary diagnoses. Both visual and statistical analyses underscored MarbliX's capability to generate meaningful compact binary barcodes, effectively discriminating between patients with distinct primary diagnoses. Furthermore, the evaluation of MarbliX in multimodal search and retrieval demonstrated its superiority over unimodal representations. Monograms consistently resulted in accurate and similar retrieved cases across various criteria, highlighting the model's capability in capturing informative patterns from both histopathology and immunogenomic data. The t-SNE maps provided visual insights into MarbliX's ability to create discriminative representations, notably enhancing the separation of cancer subtypes.

In summary, MarbliX has effectively achieved its objectives by generating informative multimodal monograms that capture and represent the complex features of patients with different cancer subtypes. Beyond demonstrating its robustness in patient representation through multimodal search, MarbliX emerges as a versatile framework with applications extending beyond search and retrieval. Its capability to efficiently integrate and encapsulate distinct data modalities, each inherently possessing large sizes and complexity, into a compact binary matrix is remarkable. This resulting binary matrix serves as a powerful bridge between different modalities, with each entry encoding whether specific features from one modality align with or correspond to certain features in the other. The binary nature of the representations simplifies storage requirements and computational complexity needed for various applications. Multimodal data fusion through MarbliX paves the way for more informed and data-driven decision-making in complex biomedical analyses.

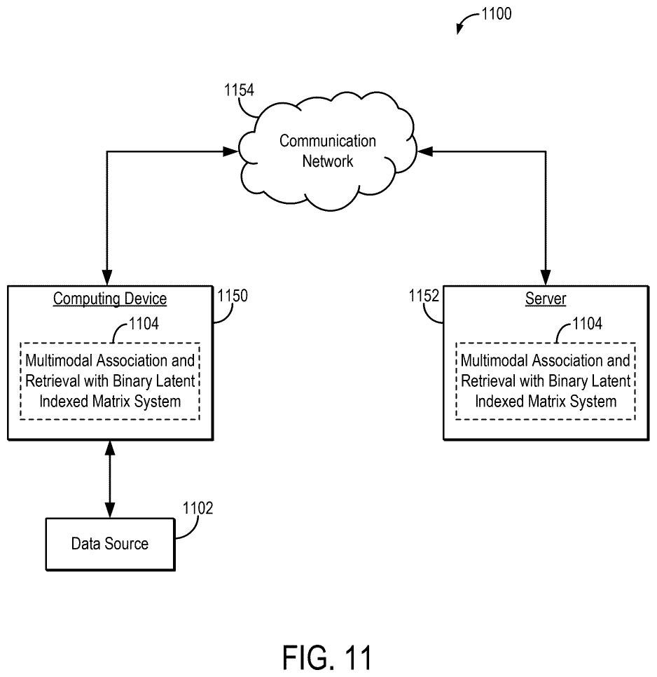

FIG. 11 shows an example of a system 1100 for generating a binary matrix representation of multimodal patient health data in accordance with some embodiments described in the present disclosure. As shown in FIG. 11, a computing device 1150 can receive one or more types of data (e.g., multimodal patient health data, which may include at least a first unimodal data subset of a first data type and a second unimodal data subset of a second data type) from data source 1102. In some embodiments, computing device 1150 can execute at least a portion of a multimodal association and retrieval with binary latent indexed matrix (MarbliX) system 1104 to generate a binary matrix representation from multimodal patient health data received from the data source 1102 and to perform downstream tasks using the binary matrix, including search and retrieval tasks across a healthcare enterprise.

Additionally or alternatively, in some embodiments, the computing device 1150 can communicate information about data received from the data source 1102 to a server 1152 over a communication network 1154, which can execute at least a portion of the MarbliX system 1104. In such embodiments, the server 1152 can return information to the computing device 1150 (and/or any other suitable computing device) indicative of an output of the MarbliX system 1104.

In some embodiments, computing device 1150 and/or server 1152 can be any suitable computing device or combination of devices, such as a desktop computer, a laptop computer, a smartphone, a tablet computer, a wearable computer, a server computer, a virtual machine being executed by a physical computing device, and so on. The computing device 1150 and/or server 1152 can also reconstruct images from the data.

In some embodiments, data source 1102 can be any suitable source of data (e.g., measurement data, images reconstructed from measurement data, processed image data, processed measurement data, other patient health data), another computing device (e.g., a server storing measurement data, images reconstructed from measurement data, processed image data, processed measurement data, other patient health data), and so on. In some embodiments, data source 1102 can be local to computing device 1150. For example, data source 1102 can be incorporated with computing device 1150 (e.g., computing device 1150 can be configured as part of a device for measuring, recording, estimating, acquiring, or otherwise collecting or storing data). As another example, data source 1102 can be connected to computing device 1150 by a cable, a direct wireless link, and so on. Additionally or alternatively, in some embodiments, data source 1102 can be located locally and/or remotely from computing device 1150, and can communicate data to computing device 1150 (and/or server 1152) via a communication network (e.g., communication network 1154).

In some embodiments, communication network 1154 can be any suitable communication network or combination of communication networks. For example, communication network 1154 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1154 can be a local area network, a wide area network, a public network (e.g., the Internet), a private or semi-private network (e.g., a corporate or university intranet), any other suitable type of network, or any suitable combination of networks. Communications links shown in FIG. 11 can each be any suitable communications link or combination of communications links, such as wired links, fiber optic links, Wi-Fi links, Bluetooth links, cellular links, and so on.

In some embodiments, the system 1100 can be configured to perform search and retrieval tasks using the binary matrix representation generated by the MarbliX system 1104. For example, the computing device 1150 and/or server 1152 can generate a search query using the binary matrix representation, search a database with the binary matrix representation, retrieve patient health data from the database based on the search query, and present the patient health data to a user via a display. The database can be stored in memory of the computing device 1150, memory of the server 1152, or in a remote data storage accessible via the communication network 1154. In some embodiments, the search can be performed using Hamming distance comparison between the query binary matrix and binary matrices stored in the database to identify similar cases.

In some embodiments, the system 1100 can be configured to perform bidirectional search using the binary matrix representation. The bidirectional search capabilities enable searches to be performed in multiple directions. For example, the computing device 1150 and/or server 1152 can search for similar images using textual data as a query, search for similar textual data or reports using imaging data as a query, or search for similar patients using a combination of imaging data and textual data as a query. In such embodiments, the processor of the computing device 1150 or server 1152 can receive a query input of one modality (e.g., textual patient health data), generate a corresponding binary matrix representation, and retrieve data of another modality (e.g., histopathology images) that share similar patterns based on the binary matrix comparison.

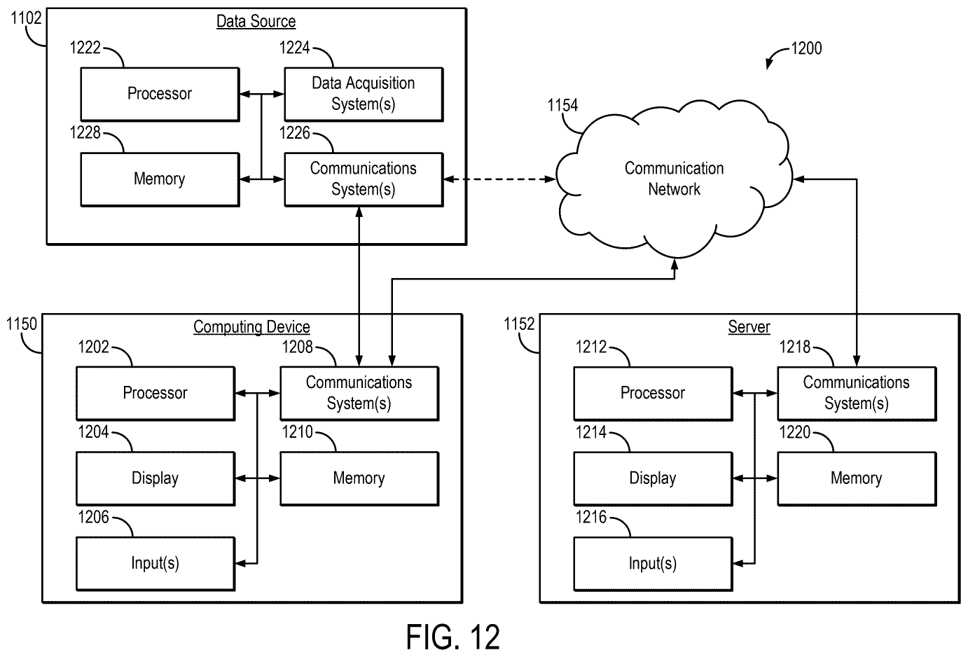

Referring now to FIG. 12, an example of hardware 1200 that can be used to implement data source 1102, computing device 1150, and server 1152 in accordance with some embodiments of the systems and methods described in the present disclosure is shown.

As shown in FIG. 12, in some embodiments, computing device 1150 can include a processor 1202, a display 1204, one or more inputs 1206, one or more communication systems 1208, and/or memory 1210. In some embodiments, processor 1202 can be any suitable hardware processor or combination of processors, such as a central processing unit (CPU), a graphics processing unit (GPU), and so on. In some embodiments, display 1204 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 1206 can include any suitable input devices and/or sensors that can be used to receive user input, such as a keyboard, a mouse, a touchscreen, a microphone, and so on.

In some embodiments, communications systems 1208 can include any suitable hardware, firmware, and/or software for communicating information over communication network 1154 and/or any other suitable communication networks. For example, communications systems 1208 can include one or more transceivers, one or more communication chips and/or chip sets, and so on. In a more particular example, communications systems 1208 can include hardware, firmware, and/or software that can be used to establish a Wi-Fi connection, a Bluetooth connection, a cellular connection, an Ethernet connection, and so on.

In some embodiments, memory 1210 can include any suitable storage device or devices that can be used to store instructions, values, data, or the like, that can be used, for example, by processor 1202 to present content using display 1204, to communicate with server 1152 via communications system(s) 1208, and so on. Memory 1210 can include any suitable volatile memory, non-volatile memory, storage, or any suitable combination thereof. For example, memory 1210 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 1210 can have encoded thereon, or otherwise stored therein, a computer program for controlling operation of computing device 1150. In such embodiments, processor 1202 can execute at least a portion of the computer program to present content (e.g., images, user interfaces, graphics, tables), receive content from server 1152, transmit information to server 1152, and so on. For example, the processor 1202 and the memory 1210 can be configured to perform the methods described herein (e.g., the workflow illustrated in FIG. 1, the method of FIG. 2, the method of FIG. 3, the method of FIG. 4, the method of FIG. 5).

In some embodiments, memory 1210 can store a database of binary matrix representations generated by the MarbliX system 1104. The processor 1202 can be configured to generate a search query using a binary matrix representation of a query case, compare the query binary matrix to binary matrices stored in the database using a distance metric such as Hamming distance, retrieve the top most similar binary matrices from the database, and present the corresponding patient health data to a user via display 1204.

In some embodiments, server 1152 can include a processor 1212, a display 1214, one or more inputs 1216, one or more communications systems 1218, and/or memory 1220. In some embodiments, processor 1212 can be any suitable hardware processor or combination of processors, such as a CPU, a GPU, and so on. In some embodiments, display 1214 can include any suitable display devices, such as an LCD screen, LED display, OLED display, electrophoretic display, a computer monitor, a touchscreen, a television, and so on. In some embodiments, inputs 1216 can include any suitable input devices and/or sensors that can be used to receive user input, such as a keyboard, a mouse, a touchscreen, a microphone, and so on.

In some embodiments, communications systems 1218 can include any suitable hardware, firmware, and/or software for communicating information over communication network 1154 and/or any other suitable communication networks. For example, communications systems 1218 can include one or more transceivers, one or more communication chips and/or chip sets, and so on. In a more particular example, communications systems 1218 can include hardware, firmware, and/or software that can be used to establish a Wi-Fi connection, a Bluetooth connection, a cellular connection, an Ethernet connection, and so on.

In some embodiments, memory 1220 can include any suitable storage device or devices that can be used to store instructions, values, data, or the like, that can be used, for example, by processor 1212 to present content using display 1214, to communicate with one or more computing devices 1150, and so on. Memory 1220 can include any suitable volatile memory, non-volatile memory, storage, or any suitable combination thereof. For example, memory 1220 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1220 can have encoded thereon a server program for controlling operation of server 1152. In such embodiments, processor 1212 can execute at least a portion of the server program to transmit information and/or content (e.g., data, images, a user interface) to one or more computing devices 1150, receive information and/or content from one or more computing devices 1150, receive instructions from one or more devices (e.g., a personal computer, a laptop computer, a tablet computer, a smartphone), and so on.

In some embodiments, the server 1152 is configured to perform the methods described in the present disclosure. For example, the processor 1212 and memory 1220 can be configured to perform the methods described herein (e.g., the workflow illustrated in FIG. 1, the method of FIG. 2, the method of FIG. 3, the method of FIG. 4, the method of FIG. 5). In some embodiments, memory 1220 can store a monogram-indexed archive comprising binary matrix representations for a plurality of patients. The processor 1212 can be configured to receive a query from computing device 1150 via communications systems 1218, perform a search of the monogram-indexed archive using the query binary matrix, and return search results to the computing device 1150. The search results can include binary matrices that share similar patterns with the query, along with associated patient health data including histopathology images, genomics data, treatment history, and risk stratification information.

In some embodiments, the server 1152 can be configured to perform bidirectional search operations. For example, the processor 1212 can receive a query comprising textual patient health data (e.g., pathology reports, clinical notes, captions describing histopathology images, image annotations, radiology reports, laboratory result narratives, or diagnostic interpretations) from the computing device 1150, generate a binary matrix representation from the textual data, search the monogram-indexed archive, and return similar histopathology images or other imaging data to the computing device 1150. Conversely, the processor 1212 can receive a query comprising imaging data (e.g., histopathology images, whole slide images), generate a binary matrix representation from the imaging data, search the monogram-indexed archive, and return similar textual data or reports to the computing device 1150. Additionally, the processor 1212 can receive a query comprising a combination of imaging data and textual data, generate a binary matrix representation from the multimodal data, and return similar patient records to the computing device 1150.

In some embodiments, data source 1102 can include a processor 1222, one or more data acquisition systems 1224, one or more communications systems 1226, and/or memory 1228. In some embodiments, processor 1222 can be any suitable hardware processor or combination of processors, such as a CPU, a GPU, and so on. In some embodiments, the one or more data acquisition systems 1224 are generally configured to acquire data, images, or both, and can include medical imaging systems, slide scanners, microscopy systems, or the like. Additionally or alternatively, in some embodiments, the one or more data acquisition systems 1224 can include any suitable hardware, firmware, and/or software for coupling to and/or controlling operations of medical imaging systems, slide scanners, microscopy systems, or the like. In some embodiments, one or more portions of the data acquisition system(s) 1224 can be removable and/or replaceable.

Note that, although not shown, data source 1102 can include any suitable inputs and/or outputs. For example, data source 1102 can include input devices and/or sensors that can be used to receive user input, such as a keyboard, a mouse, a touchscreen, a microphone, a trackpad, a trackball, and so on. As another example, data source 1102 can include any suitable display devices, such as an LCD screen, an LED display, an OLED display, an electrophoretic display, a computer monitor, a touchscreen, a television, etc., one or more speakers, and so on.

In some embodiments, communications systems 1226 can include any suitable hardware, firmware, and/or software for communicating information to computing device 1150 (and, in some embodiments, over communication network 1154 and/or any other suitable communication networks). For example, communications systems 1226 can include one or more transceivers, one or more communication chips and/or chip sets, and so on. In a more particular example, communications systems 1226 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1228 can include any suitable storage device or devices that can be used to store instructions, values, data, or the like, that can be used, for example, by processor 1222 to control the one or more data acquisition systems 1224, and/or receive data from the one or more data acquisition systems 1224; to generate images from data; present content (e.g., data, images, a user interface) using a display; communicate with one or more computing devices 1150; and so on. Memory 1228 can include any suitable volatile memory, non-volatile memory, storage, or any suitable combination thereof. For example, memory 1228 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1228 can have encoded thereon, or otherwise stored therein, a program for controlling operation of data source 1102. In such embodiments, processor 1222 can execute at least a portion of the program to generate images, transmit information and/or content (e.g., data, images, a user interface) to one or more computing devices 1150, receive information and/or content from one or more computing devices 1150, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for generating a binary matrix representation of multimodal patient health data associated with a patient, comprising:
accessing the multimodal patient health data with a computer system, wherein the multimodal patient health data comprise at least first patient health data of a first data type and second patient health data of a second data type;
accessing a machine learning model with the computer system, wherein the machine learning model is trained on training data to generate a binary matrix representation of patient health data from multimodal data;
generating a first latent representation of the first patent health data with the computer system;
generating a second latent representation of the second patient health data with the computer system;
inputting the first and second latent representations to the machine learning model using the computer system, generating a binary matrix as an output, wherein the binary matrix comprises a concise representation of the multimodal patient health data; and
outputting the binary matrix with the computer system.

2. The method of claim 1, wherein generating the first latent representation comprises:
accessing a first autoencoder network;
applying the first patient health data to the first autoencoder network; and
extracting the first latent representation from the first autoencoder network.

3. The method of claim 2, wherein the first autoencoder network has been trained on training data to reconstruct a feature vector associated with the second data type from a feature vector of the first data type.

4. The method of claim 2, wherein extracting the first latent representation from the first autoencoder network comprises extracting the first latent representation from a bottleneck layer of the first autoencoder network.

5. The method of claim 2, wherein applying the first patient health data to the first autoencoder network comprises:
generating a first feature vector from the first patient health data; and
applying the first feature vector to the first autoencoder network.

6. The method of claim 5, wherein the first feature vector is generated from the first patient health data by:
accessing a first feature extraction neural network with the computer system, wherein the first feature extraction neural network has been trained on training data to extract a feature vector from an input data set; and
applying the first patient health data to the first feature extraction neural network with the computer system, generating the first feature vector as an output.

7. The method of claim 6, wherein the first patient health data comprise imaging data and the first feature extraction neural network comprises a vision transformer.

8. The method of claim 5, wherein the first feature vector comprises a first embedding.

9. The method of claim 1, wherein generating the second latent representation comprises:
accessing a second autoencoder network;
applying the second patient health data to the second autoencoder network; and
extracting the second latent representation from the second autoencoder network.

10. The method of claim 9, wherein the second autoencoder network has been trained on training data to reconstruct a feature vector associated with the first data type from a feature vector of the second data type.

11. The method of claim 9, wherein extracting the second latent representation from the second autoencoder network comprises extracting the second latent representation from a bottleneck layer of the second autoencoder network.

12. The method of claim 9, wherein applying the second patient health data to the second autoencoder network comprises:
generating a second feature vector from the second patient health data; and
applying the second feature vector to the second autoencoder network.

13. The method of claim 12, wherein the second feature vector is generated from the second patient health data by:
accessing a second feature extraction neural network with the computer system, wherein the second feature extraction neural network has been trained on training data to extract a feature vector from an input data set; and
applying the second patient health data to the second feature extraction neural network with the computer system, generating the second feature vector as an output.

14. The method of claim 13, wherein the second patient health data comprise one of sequencing data or textual patient health data and the second feature extraction neural network comprises a language model.

15. The method of claim 14, wherein the language model is a large language model.

16. The method of claim 12, wherein the second feature vector comprises a second embedding.

17. The method of claim 1, wherein the binary matrix comprises a two-dimensional matrix.

18. The method of claim 1, wherein the first patient health data comprise imaging data.

19. The method of claim 18, wherein the imaging data comprise histopathology imaging data.

20. The method of claim 19, wherein the histopathology imaging data comprise one or more whole slide images.

21. The method of claim 19, wherein the histopathology imaging data comprise a plurality of image patches.

22. The method of claim 18, wherein the second patient health data comprise sequencing data.

23. The method of claim 22, wherein the sequencing data comprise immunogenomic sequences.

24. The method of claim 18, wherein the second patient health data comprise textual patient health data.

25. The method of claim 24, wherein the textual patient health data comprise at least one of pathology reports, clinical notes, captions describing the histopathology images, image annotations, radiology reports, laboratory result narratives, or diagnostic interpretations.

26. The method of claim 1, wherein outputting the binary matrix representation with the computer system comprises generating a search query using the binary matrix representation, searching a database with the binary matrix representation, retrieving patient health data from the database based on the search query, and presenting the patient health data to a user with the computer system.

27. The method of claim 1, wherein outputting the binary matrix representation with the computer system comprises performing a bidirectional search using the binary matrix representation.

28. The method of claim 27, wherein performing the bidirectional search comprises searching for similar images using textual data as a query.

29. The method of claim 27, wherein performing the bidirectional search comprises searching for similar textual data or reports using imaging data as a query.

30. The method of claim 27, wherein performing the bidirectional search comprises searching for similar patients using a combination of imaging data and textual data as a query.
